(19) United States
(12) Patent Application Publication
(10) Pub. No.: US 2011/0002989 A1
(43) Pub. Date: Jan. 6, 2011

(54) METHODS, DOSAGE FORMS AND KITS FOR ADMINISTERING ZIPRASIDONE WITHOUT FOOD

(75) Inventors: William John Curatolo, Niantic, CT (US); Scott Max Herbig, East Lyme, CT (US); Avinash Govind Thombre, East Lyme, CT (US); Jaymin Chandrakant Shah, Waterford, CT (US); Sheri L. Shamblin, North Stonington, CT (US); Timothy Lukas, Kent (GB); William Brett Caldwell, Bend, OR (US); Dwayne Thomas Friesen, Bend, OR (US); David Keith Lyon, Bend, OR (US); Christopher Donovan Craig, Bend, OR (US)

Correspondence Address: PFIZER INC.;PATENT DEPARTMENT, Bld 114 M/S 9114, EASTERN POINT ROAD, GROTON, CT, 06340, US

(73) Assignee: Pfizer Inc.

(21) Appl. No.: 12/920,400

(22) PCT Filed: Mar. 4, 2009
(86) PCT No.: PCT/IB09/00442

§ 371 Date: Aug. 31, 2010

Related U.S. Application Data
(60) Provisional application No. 61/034,947, filed on Mar. 7, 2008.

(51) Int. Cl. A61K 31/496; A61P 25/00; A61K 31/724; A61K 31/717; A61K 9/22

(52) U.S. Cl. 424/468; 514/254.04; 514/58; 514/25; 977/773

(57) ABSTRACT

The present invention provides methods, dosage forms and kits for treating with an effective amount of ziprasidone a CNS disorder in a human when the human is in a fasted state. In one embodiment, the invention relates to a method for treating a CNS disorder in a human, which method comprises administering to the human in a fasted state, a solid oral dosage form comprising an amount of ziprasidone effective to treat said CNS disorder, wherein the area under the serum concentration versus time curve (AUC_{0-inf}) of the ziprasidone in the human subsequent to said administering is from 70% to 140% of the mean area under the ziprasidone serum concentration versus time curve (AUC_{0-inf}) resulting from administration of a control ziprasidone immediate release oral capsule containing the same amount of ziprasidone to a cohort of humans in a fed state.

### BACKGROUND OF THE INVENTION

Food has an important impact on the absorption of many orally administered drugs (Welling P G. Effects of food on drug absorption. Annu Rev Nutr. 1996; 16:383-415). For some drugs, absorption is attenuated when taken with food. For others, food enhances absorption. Modification of drug absorption has important implications for efficacy and toxicity. Unexpectedly low absorption may manifest itself as a reduction in efficacy while higher drug absorption may result in a greater incidence of adverse events (AEs). Either consequence may influence therapeutic success and treatment compliance.

Ziprasidone (5-[2-[4-(1,2-benzisothiazol-3-yl)-1-piperazinyl]ethyl]-6-chloro-1,3-dihydro-2H-indol-2-one) is an orally active antipsychotic drug used for the monotherapy of schizophrenia (Harvey P D, Bowie C R. Ziprasidone: efficacy, tolerability, and emerging data on wide-ranging effectiveness. Expert Opin Pharmacother. 2005; 6(2):337-346) and bipolar disorder (Patel N C, Keck P E, Jr. Ziprasidone: efficacy and safety in patients with bipolar disorder. Expert Rev Neurother. 2006; 6(8):1129-1138). Following oral administration with food, peak serum ziprasidone concentrations typically occur 6 to 8 hours post-dose.

Currently, ziprasidone is approved in a solid oral capsule dosage form in the United States and many other countries and is sold under the names GEODON® Capsules (in some countries, including the United States) and ZELDOX (in other countries). An oral suspension of ziprasidone is also approved as GEODON® Oral Suspension, and an immediate-release injectable form, sold as GEODON® for Injection, is approved for treatment of acute psychotic episodes. The Label approved by the U.S. Food and Drug Association for these products states that the oral capsules should be administered with food.

Oral ziprasidone absorption is influenced by the presence of food. Studies in healthy volunteers have shown that the bioavailability of ziprasidone is enhanced when it is administered with a standard Food and Drug Administration (FDA) meal (Hamelin B A, Allard S, Laplante L, et al. The effect of timing of a standard meal on the pharmacokinetics and pharmacodynamics of the novel atypical antipsychotic agent ziprasidone. Pharmacotherapy. 1998; 18(1):9-15). Depending on the dose, there was approximately 100% enhanced absorption of ziprasidone when given with food (Miceli J J, Wilner K D, Hansen R A, Johnson A C, Apseloff G, Gerber N. Single- and multiple-dose pharmacokinetics of ziprasidone under non-fasting conditions in healthy male volunteers. Br J Clin Pharmacol. 2000; 49 Suppl 1:5S-13S). Absorption is also dependent on the timing of drug administration relative to food, with reduced absorption when taken 2 hours after, rather than immediately following, food (Hamelin et al. 1998, supra).

A typical FDA standard meal consists of 2 eggs fried in butter, 2 strips of bacon, 2 slices of toast with butter, 4 oz of hash brown potatoes and 8 oz of whole milk. The meal is high in both fat (approximately 50% of total calorie content of the meal) and calories (approximately 800-1000 kcal) (Guidance for Industry: Food-Effect Bioavailability and Fed Bioequivalence Studies. Rockville, Md.: US Department of Health and Human Services, Food and Drug Administration, Center for Drug Evaluation and Research (CDER); 2002), and is more a standardized research tool than a realistic representation of the daily diet of the anticipated patient group. In clinical practice, many patients may take ziprasidone with meals that differ substantially in calorie or fat content, and it is erroneous to assume that drug absorption in these situations will necessarily mirror that obtained under laboratory conditions.

Moreover, previous laboratory studies of ziprasidone pharmacokinetics have been acute investigations conducted in healthy volunteers rather than patients (Hamelin et al. 1998, supra; Miceli et al. 2000, supra). Patients with schizophrenia often eat a poorer diet (McCreadie R, Macdonald E, Blacklock C, et al. Dietary intake of schizophrenic patients in Nithsdale, Scotland: case-control study. BMJ. 1998; 317(7161):784-785) and do not necessarily take their medicines as instructed, raising the possibility that patients may not be receiving appropriate medication exposure where drug administration occurs outside institutional contexts.

Further, in recent market research, about 25% of physicians reported that they do not instruct their patients to take ziprasidone with food. Moreover, 50% of those psychiatrists who do instruct patients to take ziprasidone with food, told patients to take the drug with a snack. Compliance among patients was similarly poor: About 40% of patient respondents took at least half of their weekly ziprasidone doses without any calorie source.

Thus, there exists a need for a method for providing efficacious ziprasidone plasma levels to patients who would derive benefit from ziprasidone, such as patients with schizophrenia or a bipolar disorder, wherein the ziprasidone may be administered to the patients in either a fed or a fasted state. Hence, the patients would not need to worry about whether they have taken an adequate amount of food or taken any food at all in order to ensure that their ziprasidone therapy is working. The present invention addresses this need by providing ziprasidone dosage forms and methods for efficaciously administering ziprasidone which do not depend on the amount of food that a patient has ingested. This invention therefore will greatly increase patient compliance, improve patient quality of life, and consequently result in greater realized ziprasidone efficacy.

### SUMMARY OF THE INVENTION

The present invention provides a method for treating a Central Nervous System (CNS) disorder in a human, which method comprises administering to the human in a fasted state, a solid oral dosage form comprising an amount of ziprasidone effective to treat said CNS disorder, wherein the area under the serum concentration versus time curve (AUC_{0-inf}) of the ziprasidone in the human subsequent to said administering is from 70% to 140% of the mean area under the ziprasidone serum concentration versus time curve (AUC_{0-inf}) resulting from administration of a control ziprasidone immediate release oral capsule containing the same amount of ziprasidone to a cohort of humans in a fed state.

In one embodiment the serum AUC_{0-inf} of the ziprasidone in the fasted human subsequent to administering the solid oral dosage form of ziprasidone is from 75% to 130% of the mean serum AUC_{0-inf} resulting from administration of a control ziprasidone immediate release oral capsule containing the same amount of ziprasidone to a cohort of humans in a fed state. In another embodiment the serum AUC_{0-inf} of the ziprasidone in the fasted human subsequent to administering the solid oral dosage form of ziprasidone is from 80% to 125% of the mean serum AUC_{0-inf} resulting from administration of the control ziprasidone immediate release oral capsule containing the same amount of ziprasidone to a cohort of humans in a fed state.

The present invention also provides a method for treating a Central Nervous System (CNS) disorder in a human independent of whether the human is in a fasted or a fed state. Accordingly the present invention provides a method for treating a CNS disorder in a human, which method comprises administering to the human in a fasted state a solid oral dosage form comprising an amount of ziprasidone effective to treat said CNS disorder, wherein the area under the serum concentration versus time curve (AUC_{0-inf}) of the ziprasidone in the human subsequent to said administering is from 70% to 140% of the area under the ziprasidone serum concentration versus time curve that would have resulted had an identical solid oral dosage form, containing the same amount of ziprasidone, been administered to the human in a fed state. In one embodiment the AUC_{0-inf} of the ziprasidone in the fasted human is from 75% to 130% of the AUC_{0-inf} resulting from administering an identical dosage form containing the same amount of ziprasidone to a cohort of humans in a fed state. In another embodiment, the AUC_{0-inf} of the ziprasidone in the fasted human is from 80% to 125% of the mean AUC_{0-inf} resulting from administering an identical dosage form containing the same amount of ziprasidone to a cohort of humans in a fed state.

Preferably, the method provided by the present invention is one in which a CNS disorder is treated in a human by administering a solid oral dosage form comprising an effective amount of ziprasidone to the human in a fasted state, wherein the serum AUC_{0-inf} of the ziprasidone in the human subsequent to said administering is from 70% to 140%, preferably from 75% to 130%, more preferably from 80% to 125%, of the mean ziprasidone serum AUC_{0-inf} resulting from administration of a control ziprasidone immediate release oral capsule containing the same amount of ziprasidone to a cohort of humans in a fed state, and the serum AUC_{0-inf} in the human subsequent to said administering is also from 70% to 140%, preferably from 75% to 130%, more preferably from 80% to 125%, of the mean ziprasidone serum AUC_{0-inf} resulting from administering an identical solid oral dosage form, containing the same amount of ziprasidone, to a cohort of humans in a fed state.

In another aspect of the invention, the methods of the present invention provide that the maximum ziprasidone serum concentration (Cₘₐₓ) subsequent to administration in the fasted state is within about 30% of the mean maximum ziprasidone serum concentration (Cₘₐₓ) resulting from administration of a control ziprasidone immediate release oral capsule containing the same amount of ziprasidone to a cohort of humans in a fed state.

In another aspect of the invention, the methods of the present invention provide that the maximum ziprasidone serum concentration (Cₘₐₓ) subsequent to administration in the fasted state is less than about 140% of the mean maximum ziprasidone serum concentration (Cₘₐₓ) resulting from administration of a control ziprasidone immediate release oral capsule containing the same amount of ziprasidone to a cohort of humans in a fed state.

The present invention also provides a method for treating a CNS disorder in a human, which method comprises administering to the human in a fasted state a solid oral dosage form comprising an effective amount of ziprasidone providing a steady state minimum blood ziprasidone concentration (Cₘᵢₙ) of at least 20 ng/ml and a steady state maximum blood ziprasidone concentration (Cₘₐₓ) of less than 330 ng/ml.

The present invention also provides solid oral dosage forms comprising an effective amount of ziprasidone which are useful in the above-described methods. Thus, the invention also may include certain of the dosage forms that are described in the present application as being useful for achieveing any of the aforementioned methods.

In the methods and kits of the present invention, the ziprasidone in the dosage form preferably comprises ziprasidone in a form that is dissolution rate-improved and/or solubility-improved. Thus, all or a portion of the ziprasidone in the dosage form is preferably a dissolution rate-improved form of ziprasidone and/or a solubility-improved form of ziprasidone. In this context, “ziprasidone form” is distinct from the “dosage form” (as in “solid oral dosage form”). “Ziprasidone form” refers to the state of the ziprasidone ingredient that is used in the solid oral dosage form. For example a specific “ziprasidone form” may be a specific salt or hydrate of ziprasidone, a specific particle size of ziprasidone, and/or ziprasidone in combination with specific excipients. “Dosage form” refers to the shape and constitution of the dosage that is going to be administered to patients. For example a specific “dosage form” may be a tablet, capsule, or a powder for reconstitution. The dosage form can be used to modify the rate of release of the ziprasidone ingredient, for example the dissolution rate-improved or solubility-improved ziprasidone, therein.

In the methods and kits of the present invention, the solid oral dosage form useful therefore can comprise a sustained release means, a delayed release means, an immediate release portion, or any combination thereof. Preferably, in such dosage forms, the ziprasidone is in a solubility improved form or a dissolution rate improved form. The solubility improved form or dissolution rate improved form of ziprasidone may, for example, be ziprasidone in combination with a cyclodextrin, ziprasidone nanoparticles, ziprasidone tosylate, ziprasidone tartrate, or a solid mixture of ziprasidone and a polymer, in which at least a portion of the ziprasidone is semi-ordered. These forms of ziprasidone are described herein. More preferably, such solid oral dosage forms comprise a sustained release means or a delayed-release means (which means that they can also optionally comprise an immediate release portion). Further preferably, such solid oral dosage forms comprise a sustained release means (which means that they can also optionally comprise a delayed release means and/or an immediate release portion).

The sustained release means can be, for example a matrix, as in a matrix tablet. Preferably, the matrix material is HPMC.

In the methods and kits of the present invention, the solid oral dosage form useful therefore can comprise ziprasidone in combination with a precipitation inhibitor. The ziprasidone in combination with a precipitation inhibitor is preferably ziprasidone in a solubility improved form or dissolution rate improved form, for example ziprasidone in combination with a cyclodextrin, ziprasidone nanoparticles, ziprasidone tosylate, ziprasidone tartrate, or a solid mixture of ziprasidone and a polymer, in which at least a portion of the ziprasidone is semi-ordered.

In other aspects of the present invention, the methods comprise administering the oral dosage form of ziprasidone wherein ziprasidone in the dosage form has a mean particle size greater than 2000 nm. In another aspect of the present invention, the dosage form, kits and methods comprise ziprasidone having a mean particle size less than 2000 nm.

In different embodiments of the present invention, the ziprasidone solid oral dosage form includes an immediate release portion, a sustained release means, a delayed release means, or any combination thereof.

This invention also provides pharmaceutical kits for treatment of one or more CNS disorders. The kits comprise a solid oral dosage form comprising an effective amount of ziprasidone as described herein and instructions, for example a “package insert”, explaining administration of the solid oral dosage form.

In one embodiment, the present invention provides a kit comprising
a) a solid oral dosage form comprising an effective amount of ziprasidone and a pharmaceutically acceptable carrier; and
b) instructions for oral administration of the dosage form of (a), which do not specify administration with food;

wherein said solid oral dosage form when administered to a human in a fasted state provides to the human a serum ziprasidone AUC_{0-inf} which is from 70% to 140% of a mean ziprasidone serum AUC_{0-inf} resulting from administration to a cohort of humans in a fed state of a control ziprasidone immediate release oral capsule containing the same amount of ziprasidone.

In another embodiment, the present invention provides a kit comprising
a) a solid oral dosage form comprising an effective amount of ziprasidone and a pharmaceutically acceptable carrier; and
b) instructions for oral administration of the dosage form of (a), which instructions indicate that the dosage form of (a) may be administered with or without food;

wherein said solid oral dosage form when administered to a human in a fasted state provides to the human a serum ziprasidone AUC_{0-inf} which is from 70% to 140% of a mean ziprasidone serum AUC_{0-inf} resulting from administration to a cohort of humans in a fed state of a control ziprasidone immediate release oral capsule containing the same amount of ziprasidone.

In another embodiment, the present invention provides a kit comprising
a) a solid oral dosage form comprising an effective amount of ziprasidone and a pharmaceutically acceptable carrier; and
b) instructions for oral administration of the dosage form of (a), which do not specify administration with food;

wherein said solid oral dosage form when administered to a human in a fasted state provides to the human a serum ziprasidone AUC_{0-inf} which is from 70% to 140% of a mean ziprasidone serum AUC_{0-inf} resulting from administration of an identical ziprasidone solid oral dosage form containing the same amount of ziprasidone to a cohort of humans in a fed state.

In another embodiment, the present invention provides a kit comprising
a) a solid oral dosage form comprising an effective amount of ziprasidone and a pharmaceutically acceptable carrier; and
b) instructions for oral administration of the dosage form of (a), which instructions indicate that the dosage form of (a) may be administered with or without food;

wherein said solid oral dosage form when administered to a human in a fasted state provides to the human a serum ziprasidone AUC_{0-inf} which is from 70% to 140% of a mean ziprasidone serum AUC_{0-inf} resulting from administration of an identical ziprasidone solid oral dosage form containing the same amount of ziprasidone to a cohort of humans in a fed state.

The solid oral dosage forms in the kits of the present invention preferably comprise ziprasidone in a dissolution rate-improved form and/or in a solubility-improved form. The ziprasidone in the dosage form in the kits can be in combination with a precipitation inhibitor. In a preferred embodiment, the solid oral dosage form in the kits of the invention comprises a sustained release means and/or a delayed release means, optionally in combination with an immediate release portion. In another embodiment, the solid oral dosage form comprises a sustained release means, optionally in combination with a delayed release means and/or an immediate release portion.

The present invention also provides methods, kits and solid oral dosage forms for treating a CNS disorder in a human in a fasted state as described above, wherein the ziprasidone solid oral dosage form comprises a solid mixture of ziprasidone with a polymer, at least a portion of which ziprasidone is in a semi-ordered state. In one embodiment, the solid mixture comprising the semi-orderd ziprasidone contains ziprasidone having a crystal size of less than about 200 nm. In still another embodiment, the semi-ordered ziprasidone contains ziprasidone having a crystal size of from about 20 nm to less than about 200 nm.

In one embodiment, a solid mixture of ziprasidone and polymer comprising ziprasidone in semi-ordered state is prepared by

a) forming a solid amorphous dispersion comprising ziprasidone hydrochloride monohydrate or ziprasidone free base and a concentration-enhancing polymer such as HPMC or HPMCAS,

b) treating said solid amorphous dispersion to increase the mobility of said ziprasidone in said solid amorphous dispersion by heating and/or exposing said dispersion to a mobility enhancing agent, and

c) converting at least 20% of said ziprasidone to a semi-ordered state.

In a further embodiment, the solid oral dosage form is an immediate release tablet comprising a solid mixture comprising semi-ordered ziprasidone hydrochloride and hydroxypropyl methylcellulose acetate succinate (HPMCAS) as described in WO 2004/014342 A1. In another embodiment, the solid oral dosage form is a sustained release tablet comprising a solid mixture comprising semi-ordered ziprasidone hydrochloride and HPMCAS. In one embodiment comprising a solid mixture comprising semi-ordered ziprasidone hydrochloride and polymer (for example, HPMCAS), the solid oral dosage form is a sustained release matrix tablet, preferably comprising HPMC as matrix material. In such embodiment, the sustained release matrix tablet preferably releases no more than 50 wt % of the ziprasidone therein within the first ½ hour, more preferably no more than 50 wt % within the first hour, after introduction of the tablet to an aqueous environment of use. In another embodiment, the solid oral dosage form is an immediate release tablet comprising a solid dispersion comprising semi-ordered ziprasidone free base and hydroxypropyl methylcellose (HPMC). In still another embodiment, the solid oral dosage form comprises an immediate-release portion and a sustained release portion, wherein the sustained release portion comprises a solid dispersion comprising semi-ordered ziprasidone hydrochloride and HPMCAS.

In another embodiment, the solid oral dosage form is a sustained release matrix tablet comprising a solid dispersion comprising semi-ordered ziprasidone hydrochloride and HPMCAS, preferably comprising HPMC as the matrix material, and wherein the matrix tablet releases no more than 50 wt % of the ziprasidone originally present in the tablet within ½ hour after introduction of the tablet to an aqueous environment of use, preferably within 1 hour.

In still another embodiment, the solid oral dosage form is an immediate release tablet comprising a solid dispersion comprising ziprasidone and a cyclodextrin, as described in U.S. Pat. No. 5,134,127. In another embodiment, the solid oral dosage form is an immediate release tablet comprising a solid dispersion comprising ziprasidone and a cyclodextrin, as described in U.S. Pat. Nos. 6,232,304; 5,874,418; and 5,376,645. In still another embodiment, the solid oral dosage form is sustained release tablet comprising a solid dispersion comprising ziprasidone and a cyclodextrin, as described in U.S. Pat. No. 5,134,127. In another embodiment, the solid oral dosage form is a sustained release tablet comprising a solid dispersion comprising ziprasidone and a cyclodextrin, as described in U.S. Pat. Nos. 6,232,304; 5,874,418; and 5,376,645. In one embodiment, the solid oral dosage form is a sustained release matrix tablet. In a preferred embodiment, the matrix material comprises HPMC. In still another embodiment, the solid oral dosage form comprises an immediate-release portion and a sustained release portion, wherein the sustained release portion comprises a solid dispersion comprising ziprasidone and a cyclodextrin.

In another embodiment, ziprasidone nanoparticles are prepared by
a) preparing a suspension of ziprasidone nanoparticles by techniques well known in the art such as attrition milling or high pressure homogenization,
b) lyophilizing the suspension or spray drying the suspension to form solid ziprasidone nanoparticles.

In a further embodiment, the solid oral dosage form is a sustained release matrix tablet comprising ziprasidone nanoparticles, preferably comprising HPMC as the matrix material, and wherein the matrix tablet releases no more than 50 wt % of the ziprasidone originally present in the tablet within ½ hour after introduction of the tablet to an aqueous environment of use, preferably within 1 hour. In another embodiment, the solid oral dosage form comprises an immediate release portion and a sustained release portion, wherein the sustained release portion comprises ziprasidone nanoparticles.

In another embodiment, the solid oral dosage form is a capsule containing beads coated with particles of solid dispersion comprising semi-ordered ziprasidone and polymer. In one embodiment, the particles of solid dispersion are obtained by milling a solid dispersion comprising semi-ordered ziprasidone and polymer. In another embodiment the particles of solid dispersion are obtained from a solid dispersion of semi-ordered ziprasidone hydrochloride and polymer or semi-ordered ziprasidone free base and polymer. In another embodiment the polymer is HPMCAS or HPMC. In another embodiment, the particles of solid dispersion of semi-ordered ziprasidone and polymer are coated with an enteric coating.

In another embodiment, the solid oral dosage form is a tablet containing beads coated with particles of solid dispersion comprising semi-ordered ziprasidone and polymer. The tablet is prepared by compressing pharmaceutically acceptable excipients and beads coated with particles of solid dispersion comprising semi-ordered ziprasidone and polymer. In another embodiment, the beads coated with the solid dispersion particles are further coated with an enteric coating.

In another embodiment of the invention the solid oral dosage form, and the methods and kits comprising same, comprises ziprasidone tosylate or ziprasidone tartrate. In such embodiments, the solid oral dosage form may for example be an immediate-release tablet. As another example, the solid oral dosage form comprising ziprasidone tosylate or ziprasidone tartrate may be a sustained release matrix tablet, preferably comprising HPMC as matrix material. As another example, the solid oral dosage form comprising ziprasidone tosylate or ziprasidone tartrate comprises beads coated with the ziprasidone tosylate or ziprasidone tartrate. The beads coated with the ziprasidone tartrate or ziprasidone tosylate may optionally be further coated with an enteric coating.

### BRIEF DESCRIPTION OF THE DRAWING

FIG. 1: FIG. 1 shows in vitro dissolution test results for three different sustained release matrix tablets (Examples 20, 21 and 22) comprising a solid dispersion of ziprasidone hydrochloride and HPMCAS, wherein the ziprasidone is in a semi-ordered state.

FIG. 2: In vitro dissolution test results for Examples 23, 24 and 25 (sustained release matrix tablets containing ziprasidone crystallized spray-dried dispersion) in 0.05M NaH₂PO₄ media with the addition of 2% (w/v) sodium dodecyl sulfate (SDS) and adjusted to a pH of 7.5, paddles at 75 rpm.

FIG. 3: In vitro dissolution test results for Formulation D1 (Example 26), a tablet containing ziprasidone crystallized spray-dried dispersion.

FIG. 4: In vitro dissolution test results for Form B6 (Example 28), beads coated with ziprasidone crystallized spray-dried dispersion with Eudragit® enteric coating.

FIG. 5: In vitro dissolution test results for Form B5 (Example 29), beads coated with ziprasidone crystallized spray-dried dispersion with HPMCAS enteric coating.

### DETAILED DESCRIPTION

Ziprasidone is 5-[2-[4-(1,2-benzisothiazol-3-yl)-1-piperazinyl]ethyl]-6-chloro-1,2-dihydro-2H-indol-2-one, a known compound having the structure:

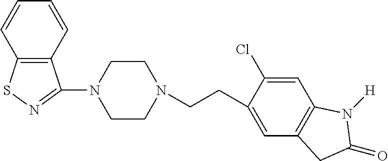

Ziprasidone and methods for synthesizing it are disclosed in numerous patents, including U.S. Pat. Nos. 4,831,031 and 5,312,925, both of which are herein incorporated by reference in their entirety. Ziprasidone has utility as a neuroleptic, and is thus useful, inter alia, as an antipsychotic. Ziprasidone is typically administered in a daily dose of from 40 mgA to 160 mgA, depending on patient need. By “daily dose” is meant the total amount of mgA ziprasidone administered to a patient in one day.

The term “ziprasidone” should be understood herein, unless otherwise indicated herein, to include any pharmaceutically acceptable form of the compound. By “pharmaceutically acceptable form” is meant any pharmaceutically acceptable derivative or variation, including, solvates, hydrates, isomorphs, polymorphs, pseudomorphs, neutral forms, acid addition salt forms, and prodrugs. Ziprasidone may be present in crystalline or amorphous form. The pharmaceutically acceptable acid addition salts of ziprasidone are prepared in a conventional manner by treating a solution or suspension of the free base with about one chemical equivalent of a pharmaceutically acceptable acid. Conventional concentration and recrystallization techniques are employed in isolating the salts. Illustrative of suitable acids are acetic, lactic, succinic, maleic, tartaric, citric, gluconic, ascorbic, mesylic, tosylic, benzoic, cinnamic, fumaric, sulfuric, phosphoric, hydrochloric, hydrobromic, hydroiodic, sulfamic, sulfonic such as methanesulfonic, benzenesulfonic, and related acids. Preferred forms of ziprasidone include the free base, ziprasidone hydrochloride monohydrate, ziprasidone mesylate trihydrate, ziprasidone tartrate, and ziprasidone tosylate.

The “solid oral dosage form” of the present invention is a pharmaceutically-acceptable solid oral dosage form, meaning that the dosage form is safe for administration to humans and all excipients in the dosage form are pharmaceutically-acceptable, in other words safe for human ingestion.

The phrase “fasted state” as used herein, unless otherwise indicated, in reference to a human or other mammal, means that the human or other mammal has not ingested 500 calories or more than 500 calories for at least two hours before ingesting the ziprasidone solid oral dosage form and for at least two hours after ingesting the ziprasidone solid oral dosage form. Preferable, “fasted state” refers to a human who has not ingested 250 calories or more than 250 calories for at least two hours before ingesting the ziprasidone solid oral dosage form and, for at least two hours after ingesting the ziprasidone solid oral dosage form, will not ingest more than 250 calories. For example, to illustrate, a human in a “fasted state” may have ingested zero calories within the two hours before dosing, and will only ingest 100 calories during the two hour period after dosing.

The term “fed state” as used herein, unless otherwise indicated, refers to a human or other mammal that has ingested at least 500 calories within the time period consisting of the two hours prior to dosing and the two hours subsequent to dosing of the ziprasidone solid oral dosage form of the invention. In another embodiment, “fed state” refers to a human or other mammal that has ingested at least 800 calories within said time period. In another embodiment, “fed state” refers to a human or other mammal that has ingested at least 1000 calories within said time period. In still another embodiment, “fed state” refers to a human who has eaten a United States Food and Drug Administration (FDA) standard high fat breakfast (or other meal containing a comparable quantity of fat and calories) within said time period. A typical FDA standard breakfast consists of 2 eggs fried in butter, 2 strips of bacon, 2 slices of toast with butter, 4 ounces of hash brown potatoes and 8 ounces of whole milk. The meal is high in both fat (approximately 50% of total calorie content of the meal) and calories (approximately 800-1000 calories).

The ziprasidone AUC_{(0-inf)} and Cₘₐₓ that will be obtained in a fasted human subsequent to administering any particular ziprasidone oral dosage form to the fasted human can be determined by means of testing that dosage form in a cohort of human subjects in a clinical study. In other words, the ziprasidone AUC and Cₘₐₓ that will be provided to an individual human by administration of any particular ziprasidone dosage form can be predicted by or defined as the mean AUC and Cₘₐₓ obtained from conducting a clinical study on a cohort of humans with that particular dosage form. The clinical study can be conducted according to guidelines issued by the U.S. Food and Drug Administration (U.S. FDA) on Food-Effect Bioavailability and Fed Bioequivalence Studies.

In such clinical study, the test ziprasidone solid oral dosage form can be administered to a cohort of humans who have not eaten any food for at least ten hours and will not eat any food for at least four hours after administration of the dosage form. For comparison's sake, another cohort of humans can be administered a control ziprasidone immediate release oral capsule containing the same amount of ziprasidone as the test dosage form, or an identical ziprasidone dosage form to the test ziprasidone solid oral dosage form, in a fed state, for example about 30 minutes after they begin eating a United States Food and Drug Administration (FDA) standard high fat breakfast, or other meal containing a quantity of fat and calories comparable thereto, the cohort completing eating the breakfast or other meal within about 30 minutes or less of beginning the breakfast or other meal. The cohorts can subsequently be “switched”, with the cohort which had been tested with the fasted protocol being tested according to the fed protocol, and the cohort which had been tested with the fed protocol being tested according to the fasted protocol. It is recommended that a period of time, sometimes referred to as a “washout period”, of at least seven days from completion of the first dosing pass before switching the fed or fasted protocol for each cohort.

The calculation of the mean area under the serum concentration versus time curve (AUC) is a well-known procedure in the pharmaceutical arts and is described, for example, in Welling, “Pharmacokinetics Processes and Mathematics,” ACS Monograph 185 (1986).

The “control ziprasidone immediate release oral capsule” referred to herein, unless otherwise indicated, is the GEODON/ZELDOX™ capsules for oral administration available commercially manufactured by Pfizer, Inc. or an equivalent thereto which would useful for comparing a solid oral dosage form of ziprasidone. GEODON/ZELDOX™ commercial capsules contain crystalline ziprasidone hydrochloride monohydrate having a Volume Mean Diameter (VMD) particle size of less than about 40 microns. GEODON/ZELDOX™ capsules also contain lactose, pregelatinized starch, and magnesium stearate. Capsules of such type are described in, for example, Table 1 of U.S. Pat. No. 6,150,366, which is incorporated herein by reference in its entirety. For example, a capsule as described in Table 1 of U.S. Pat. No. 6,150,366 containing ziprasidone hydrochloride monohydrate having a VMD of about 20 microns as described therein can be used as a control ziprasidone immediate release oral capsule for purposes of the present invention.

VMD refers to the diameter of a spherical particle having a volume which is the average volume of all particles (such as ziprasidone particles) in a sample, estimating their volume based on an assumption of spherical shape. Particle size distribution can be measured by Malvern light scattering as known to those skilled in the art.

The subject invention, as explained above, also relates to treating a CNS disorder in a human by using a ziprasidone solid oral dosage form which provides a ziprasidone AUC when the human is in a fasted state which is 70% to 140% of the ziprasidone AUC provided by an identical ziprasidone solid oral dosage form when a human is in a fed state. In this regard, the “identical” ziprasidone solid oral dosage form refers to a ziprasidone solid oral dosage form containing an identical amount of ziprasidone, in an identical ziprasidone formulation (e.g. the same salt form and particle size and including the same excipients, if any, in the same amounts), and in an identical dosage form (e.g. including the same excipients in the same amounts).

In general, a “human” for purposes of the present invention refers to a subject at least twelve years of age.

The ziprasidone in the serum of a human may be detected and measured using methods known in the art. For example, ziprasidone may be detected and measured as described in Miceli et al. Pharmacokinetics, Safety, and Tolerability of Intramuscular Ziprasidone in Healthy Volunteers, J Clin Pharmacol 2005; 45:620-630; and in Janiszewski et al. Development and Validation of a High-senstivity Assay for an Antipsychotic Agent, CP-88.059, with Solid-phase Extraction and Narrow-bore, High-performance Liquid Chromatography, J Chromatogr. 1995; 668:133-139.

As used herein, the terms “mgA” and “μgA” and “wt % A” mean milligrams, micrograms, and weight percent of active ziprasidone, respectively, wherein “active” ziprasidone refers to the non-salt, non-hydrated free base form of ziprasidone, having a molecular weight of 412.94 g/mole.

### Dissolution Rate-Improved Forms and Solubility-Improved Forms of Ziprasidone:

The ziprasidone in the dosage forms, methods, and kits of the present invention preferably comprises ziprasidone in a dissolution rate-improved form or a solubility-improved form. The inventors have found that by using a form of ziprasidone that dissolves in a use environment at a sufficiently fast rate, a rate at least faster than the ziprasidone in the control immediate release oral capsules known as GEODON/ZELDOX™, or has a high enough solubility in a use environment, at least a higher solubility than the solubility of ziprasidone in GEODON/ZELDOX™ immediate release capsules, or both, the requirement to take ziprasidone with food, for example with about 500 calories or more, in order to obtain an effective systemic ziprasidone exposure is eliminated. Further, using such dissolution rate-improved or solubility-improved form, the dosage forms used in the present invention may be taken without food, for example with about 250 calories or less (for example, zero calories) or they may be taken with food.

As discussed above, GEODON/ZELDOX™ immediate release capsules contain crystalline ziprasidone hydrochloride monohydrate having a VMD particles size of about 40 microns or less, preferably from about 5 microns to about 30 microns. Accordingly, a “solubility-improved form” of ziprasidone is a form of ziprasidone that has a solubility in a use environment that is greater than the solubility of crystalline ziprasidone hydrochloride monohydrate having a VMD particle size of about 40 microns or less, preferably about 5 microns to about 30 microns, in the use environment. A “dissolution rate-improved form” of ziprasidone is a form of ziprasidone that dissolves in a use environment at a faster rate than the dissolution rate of crystalline ziprasidone hydrochloride monohydrate having a VMD particles size of about 40 microns or less, preferably about 5 microns to about 30 microns, in the use environment. A form of ziprasidone may be both solubility-improved and dissolution-rate improved.

Preferably, the ziprasidone form is at least about 1.25 fold greater in dissolution rate or solubility than the form of ziprasidone in the control GEODON/ZELDOX™ immediate release capsules. A ziprasidone form with an even greater dissolution rate or solubility than the form of ziprasidone in the control GEODON/ZELDOX™ immediate release capsules can be utilized. Thus, the ziprasidone form may be about 2-fold, 3-fold, 5-fold, 10-fold or more the dissolution rate or solubility of the form of ziprasidone in the control GEODON/ZELDOX™ immediate release capsules.

Solubility improved forms of ziprasidone may include, without limitation, certain salts, amorphous forms, nanocrystalline forms, semi-ordered drug forms, solid amorphous dispersion forms, adsorbed drug forms, cyclodextrin and drug forms, and self-emulsifying forms.

Dissolution rate improved forms may include, without limitation, solubility improved forms as described above alone or in combination with precipitation inhibitors, crystalline forms with reduced particle size alone or in combination with a precipitation inhibitor, solubility improved forms as described above that have reduced particle size alone or with one or more precipitation inhibitors.

The use environment may be an in vitro or in vivo use environment, such as the gastrointestinal (GI) tract of a mammal, including but not limited to a human. The in vivo use environment is an aqueous environment and may be any location within the GI tract, for example within the stomach or any part of intestine. The in vitro use environment may be an environment designed to model a location within a mammalian GI tract and, accordingly is aqueous. Examples of dissolution test media which can serve as an in vitro use environment for the present invention include phosphate buffered saline (PBS) solution, model fasted duodenal (MFD) solution, simulated gastric and simulated intestinal buffer solution, and water. An appropriate PBS solution is an aqueous solution comprising 20 mM Na₂HPO₄, 47 mM KH₂PO₄, 87 mM NaCl, and 0.2 mM KCl, adjusted to pH 6.5 with NaOH. An appropriate MFD solution is the same PBS solution wherein there is also present 7.3 mM sodium taurocholic acid and 1.4 mM of 1-palmitoyl-2-oleyl-sn-glycero-3-phosphocholine. An appropriate simulated gastric buffer solution is 0.01 N HCl solution at pH 2.0. Appropriate simulated intestinal buffer solutions include (1) 50 mM NaH₂PO₄ and 2 wt % sodium lauryl sulfate, adjusted to pH 7.5, (2) 50 mM NaH₂PO₄ and 2 wt % sodium lauryl sulfate, adjusted to pH 6.5, and (3) 6 mM NaH₂PO₄, 150 mM NaCl, and 2 wt % sodium lauryl sulfate, adjusted to pH 6.5.

An example of an assay that can be used to determine if a ziprasidone form is a dissolution rate-improved form or a solubility-improved form for purposes of the present invention is an in vitro dissolution test as follows:

An in vitro dissolution test may be performed by adding the test form of ziprasidone to a dissolution test media consisting of a simulated gastric buffer (GB) solution of 0.01 N HCl at pH 2.0. It is important to note that the proposed dissolution rate-improved or solubility-improved ziprasidone form is dissolution tested independently of the dosage form so that the dosage form does not interfere with evaluation of the degree of dissolution rate or solubility, improvement.

In such a test, two end-points are assessed: 1) the maximum dissolved drug concentration (MDC) of ziprasidone in the test media, and 2) the area under the concentration versus time curve of dissolved ziprasidone in the in vitro dissolution test. More specifically, in the in vitro use environment, the concentration of dissolved ziprasidone over any 90-minute period from about 0 to about 270 minutes following introduction to the use environment is measured.

An in vitro test to evaluate enhanced ziprasidone dissolution rate in aqueous solution can be conducted by adding with agitation a sufficient quantity of test ziprasidone form to the test medium, such that if all the ziprasidone dissolved, the theoretical concentration of ziprasidone would exceed the equilibrium concentration provided by the control ziprasidone form by a factor of at least 2, and preferably by a factor of at least 10. Performing the test at this concentration will ensure that the MDC of ziprasidone can be determined.

Then, the concentration of dissolved ziprasidone is measured as a function of time by sampling the test medium and plotting ziprasidone concentration in the test medium versus time so that the dissolution rate can be ascertained. The MDC is taken to be the maximum value of dissolved ziprasidone measured over the duration of the test. The aqueous AUC may be calculated by integrating the concentration versus time curve over any 90-minute time period between the time of introduction of the composition into the aqueous use environment (when time equals zero) and 270 minutes following introduction to the use environment (when time equals 270 minutes). Typically, when the composition reaches its MDC rapidly, (in less than about 30 minutes), the time interval used to calculate AUC is from time equals zero to time equals 90 minutes.

During such a test, the concentration of “dissolved drug” should be measured using standard analytical techniques, including high-performance liquid chromatography (HPLC), ultraviolet (UV) absorption, or other standard methods known in the art. When using solution techniques, such as HPLC, the test solution should be either filtered or centrifuged, to avoid large drug particulates that would give an erroneous determination.

“Dissolved drug” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40%) than that obtained with the filter specified above but will still allow identification of preferred dissolution rate-improved forms. When measuring the concentration by UV, one should take precautions to ensure other excipients in the dissolution rate-improved formulation do not interfere with the ziprasidone UV absorbance, as known by one skilled in the art. The inventors have found that UV probes are effective at determining the concentration of dissolved ziprasidone in in vitro dissolution test media.

A specific example of a dissolution test for ascertaining solubility and dissolution rate of a test ziprasidone form is as follows: First, a sufficient amount of the test form is placed into a dissolution flask such that the concentration of ziprasidone would have been 200 μgA/mL if all the ziprasidone dissolved. A simulated GB solution at pH 2.0 is then added and the mixture stirred at a stirred speed of 100 rpm. The test is performed at 37° C. Using UV probes, the concentration of dissolved ziprasidone is then measured over time for at least 90 minutes. The formulation is considered to be a solubility-improved form or dissolution rate-improved form of ziprasidone if it provides at least one of (1) a MDC that is greater than that of the control, (2) an AUC₉₀ that is greater than that of the control, or (3) both (1) and (2). The control formulation, as discussed above, is crystalline ziprasidone HCl monohydrate as is used in the GEODON/ZELDOX™ commercial capsules.

In one embodiment, the form of ziprasidone used in the invention is a salt form of ziprasidone. It is known that some low-solubility drugs may be formulated in highly soluble salt forms that provide temporary improvements in the concentration of the drug in a use environment relative to another salt form of the drug. Examples of such salt forms for ziprasidone are the tosylate and the tartrate salt. These salts have a higher sustained solubility in gastric buffer medium as exemplified in the examples. In another embodiment, the form of ziprasidone comprises ziprasidone having a volume weighted mean particle size of less than about 10 microns and preferably less than about 5 microns. Standard crystalline ziprasidone HCl is typically in block or needle habits. The size of such crystals is commonly 30 microns long and 4 microns wide, but there is a wide range observable. When these crystals are analyzed by a Malvern Mastersizer and studied as a wet slurry, the volume-weighted mean diameter is about 10 microns. Reducing the particle size of ziprasidone improves its dissolution rate, thus providing at least temporarily enhanced concentrations of dissolved ziprasidone in an aqueous use environment relative to the concentration achieved with larger crystal sizes. Such small particles may be achieved by conventional grinding and milling techniques. In one preferred process, the ziprasidone is jet milled. Jet-milled ziprasidone may have a volume weighted mean diameter of less than about 5 microns, and preferably less than about 3 microns.

In another embodiment, the ziprasidone may be in the form of nanoparticles. The term “nanoparticle” refers to ziprasidone in the form of particles generally having an effective average crystal size of less than about 2000 nm, more preferably less than about 1000 nm. In another embodiment, the ziprasidone particles are around 500 nm or less. More preferably, the ziprasidone nanoparticles are from about 120 nm to about 400 nm. In still another embodiment, the ziprasidone nanoparticles are from about 22 nm to about 350 nm. In another embodiment the ziprasidone nanoparticles are about 250 nm or less than about 250 nm. In another embodiment, the ziprasidone nanoparticles are about 100 nm or less than about 100 nm. The ziprasidone nanoparticles may comprise one or more surface stabilizers. Ziprasidone nanoparticles and methods for preparation thereof are described in WO 2006/109183 and WO 2006/109177, both incorporated herein by reference in their entireties.

Yet another form of ziprasidone useful in the present invention is ziprasidone in amorphous form. Preferably, at least a major portion of the ziprasidone is amorphous. By “amorphous” is meant simply that the ziprasidone is in a non-crystalline state. As used herein, the term “a major portion” of means that at least 60 wt % of the drug in the dosage form is in the amorphous form, rather than the crystalline form. Preferably, the ziprasidone is substantially amorphous. As used herein, “substantially amorphous” means that the amount of ziprasidone in crystalline form does not exceed about 25 wt %. More preferably, the ziprasidone is “almost completely amorphous,” meaning that the amount of ziprasidone in the crystalline form does not exceed about 10 wt %. Amounts of crystalline ziprasidone may be measured by Powder X-Ray Diffraction (PXRD), Scanning Electron Microscope (SEM) analysis, differential scanning calorimetry (DSC), or any other standard quantitative measurement.

Examples of amorphous forms of ziprasidone include solid amorphous dispersions of ziprasidone in a polymer, such as disclosed in commonly assigned US published patent application 2002/0009494A1 herein incorporated by reference. In another embodiment, ziprasidone may be adsorbed in amorphous form on a solid substrate, such as disclosed in commonly assigned US published patent application 2003/0054037A1, herein incorporated by reference. In yet another embodiment, amorphous ziprasidone may be stabilized using a matrix material, such as disclosed in commonly assigned US Patent application 2003/0104063A1, herein incorporated by reference.

In a solid amorphous dispersion comprising ziprasidone, the polymer used in the molecular dispersion may be any pharmaceutically acceptable polymer. The term “polymer” is used conventionally, meaning a compound that is made of monomers connected together to form a larger molecule. A polymer generally consists of at least about 20 monomers connected together. Thus, the molecular weight of the polymer generally will be about 2000 daltons or more. The polymer should be inert, in the sense that it does not chemically react with the ziprasidone in an adverse manner, and should be pharmaceutically acceptable. Exemplary polymers include hydroxypropyl methyl cellulose acetate succinate (HPMCAS), hydroxypropyl methyl cellulose phthalate (HPMCP), hydroxypropyl methyl cellulose (HPMC), cellulose acetate phthalate (CAP), cellulose acetate trimellitate (CAT), carboxymethyl ethylcellulose (CMEC), poloxamers (also known as polyoxyethylene-polyoxypropylene block copolymers), polyvinyl pyrrolidone (PVP), and mixtures thereof. In one embodiment, the polymer is HPMCAS. At least a major portion of the drug in the dispersion is amorphous. Preferably, the drug in the dispersion is “substantially amorphous,” meaning that the amount of the drug in crystalline form does not exceed about 25%. More preferably, the drug in the dispersion is “almost completely amorphous,” meaning that the amount of drug in the crystalline form does not exceed about 10%. The amount of ziprasidone relative to the amount of polymer present in the solid amorphous dispersion may vary widely from a drug-to-polymer weight ratio of from 0.01 to about 4 (e.g., 1 wt % drug to 80 wt % drug). In one embodiment, the drug-to-polymer ratio is greater than about 0.05 (4.8 wt % drug) and no more than about 3 (75 wt % drug). In another embodiment, the drug-to-polymer ratio ranges from 0.11 (10 wt % drug) to 2 (67 wt % drug). In another embodiment, the drug-to-polymer ratio ranges from 0.11 (10 wt % drug) to 1 (50 wt % drug). In another embodiment, the drug-to-polymer ratio ranges from 0.15 (13 wt % drug) to 0.7 (41 wt % drug). In still another embodiment, the drug-to-polymer ratio ranges from 0.15 (13 wt % drug) to 0.6 (37.5 wt % drug).

The amorphous drug can exist within the solid amorphous dispersion as a pure phase, as a solid solution of drug homogeneously distributed throughout the polymer or any combination of these states or those states that lie between them. In one embodiment, at least a portion of the amorphous drug and polymer are present as a solid solution. This may be shown by the presence of at least one glass transition temperature for the solid amorphous dispersion that is intermediate that of the pure drug and pure polymer. In another embodiment, the dispersion is substantially homogeneous so that the amorphous drug is dispersed as homogeneously as possible throughout the polymer. As used herein, “substantially homogeneous” means that the fraction of drug present in relatively pure amorphous domains within the solid dispersion is relatively small, on the order of less than 20%. In still another embodiment, the dispersion is completely homogeneous, meaning the amount of drug in pure amorphous domains is less than 10% of the total amount of drug.

Solid amorphous dispersions may be made by a solvent-based process as follows. A feed solution is formed comprising the drug, a polymer, and a solvent. The solvent is then rapidly removed from the feed solution to form particles of drug and polymer. Suitable processes for rapidly removing the solvent include spray-drying, spray-coating, and evaporation. Further details of the spray-drying process for forming solid amorphous dispersions are disclosed in US published patent application 2002/0009494A1, supra.

In another form useful for the present invention, the ziprasidone is in a semi-ordered state, such as disclosed in WO 2004/014342, herein incorporated by reference. In such embodiment, the ziprasidone is present in a solid mixture with a polymer wherein at least a portion of the ziprasidone is “semi-ordered.” By “semi-ordered” is meant that (1) the ziprasidone is less ordered than ziprasidone in bulk crystalline form alone and (2) the ziprasidone has greater order than amorphous drug. The semi-ordered state may be in the form of extremely small crystals (e.g., less than about 200 nm), crystalline ziprasidone which has polymer incorporated into the crystals, crystals containing a multitude of crystal defects, or semi-crystalline structures which take the form of sheets, tubes, or other structures in which the ziprasidone is ordered but is not in the lowest solubility, bulk crystalline form alone. In one embodiment, the semi-ordered ziprasidone has crystals that are less than about 500 nm. In another embodiment, the semi-ordered ziprasidone has crystals that are less than about 400 nm. In still another embodiment, the semi-ordered ziprasidone has crystals that are less than about 200 nm. In still another embodiment, the semi-ordered ziprasidone has crystals that are from about 20 nm to less than about 200 nm. Ziprasidone that is semi-ordered exhibits physical characteristics that are distinct from both bulk crystalline ziprasidone (that is, crystalline ziprasidone having a volume mean diameter [VMD] of about 40 μm or less) and amorphous ziprasidone. That the ziprasidone is semi-ordered may be demonstrated by conventional techniques used to characterize whether a material is crystalline or amorphous. In one embodiment, ziprasidone is semi-ordered if the composition exhibits a powder x-ray diffraction pattern having at least one peak that has a full width at half height of at least 1.1-fold that of an equivalent peak exhibited by sziprasidone in bulk crystalline form having a VDM of about 40 μm or less. The full-width at half-height may be even broader, and may be at least 1.25-fold, 2-fold or 3-fold or greater that of the corresponding principal peak of drug in bulk crystalline form alone. In such particles, at least a portion of the ziprasidone, a portion of the polymer, or both are in a non-crystalline state. The polymer can be virtually any polymer, such as the polymers listed above for solid amorphous dispersions.

One method to form compositions containing semi-ordered ziprasidone is to first form a solid amorphous dispersion, as previously described. The dispersion is then exposed to a mobility-enhancing agent, such as water, and then treated, such as with heat, to convert at, least a portion of the amorphous ziprasidone in the dispersion into the semi-ordered state. When made in this manner, semi-orderd ziprasidone cmpositions are also referred to as crystallized spray dried dispersions (CSDD). In one embodiment, the solid amorphous dispersion is heated to a temperature T such that T_{g}/T is less than or equal to about 1.0, wherein the T_{g} is a glass transition temperature of the solid amorphous dispersion in the presence of the mobility enhancing agent, and T and T_{g} are measured in Kelvin. In another embodiment, the solid amorphous dispersion is exposed to water at a temperature above the glass-transition of the solid amorphous dispersion in the presence of the water. Details of methods for making semi-ordered drugs and techniques for verifying that the ziprasidone is in a semi-ordered state (including PXRD, spectroscopic analysis and thermal techniques) are disclosed in WO 2004/014342, supra.

Another form of ziprasidone useful in the present invention comprises ziprasidone combined with a cyclodextrin. The ziprasidone and the cyclodextrin can be in a variety of combinations with one another. For example the ziprasidone and the cyclodextrin may form an inclusion complex. In another example, the ziprasidone and the cyclodextrin are in a physical, mixture with one another. Another example of a combination of ziprasidone and a cyclodextrin are where the ziprasidone and the cyclodextrin are in a dispersion with one another, for example a spray-dried dispersion (SDD). As used herein, the term “cyclodextrin” refers to all forms and derivatives of cyclodextrin. Particular examples of cyclodextrin include α-cyclodextrin, β-cyclodextrin, and γ-cyclodextrin. Exemplary derivatives of cyclodextrin include mono- or polyalkylated β-cyclodextrin, mono- or polyhydroxyalkylated β-cyclodextrin, such as hydroxypropyl β-cyclodextrin (hydroxypropylcyclodextrin), mono, tetra or hepta-substituted β-cyclodextrin, and sulfoalkyl ether cyclodextrins (SAE-CD), such as sulfobutylether cyclodextrin (SBECD).

Simple physical mixtures of ziprasidone and cyclodextrin are described in, for example, U.S. Pat. No. 5,134,127, herein incorporated by reference. Alternatively, the drug can be formulated by using a film coating surrounding a solid core comprising a release rate modifier and a SAE-CD/drug mixture, as disclosed in U.S. Pat. No. 6,046,177, herein incorporated by reference. Alternatively, formulations containing SAE-CD may consist of a core comprising a physical mixture of one or more SAE-CD derivatives, an optional release rate modifier, a therapeutic agent, a major portion of which is not complexed to the SAE-CD, and an optional release rate modifying coating surrounding the core. Other cyclodextrin/drug forms contemplated for use in the invention are found in U.S. Pat. Nos. 6,232,304; 5,874,418; and 5,376,645, all of which are incorporated herein by reference.

Another useful form of ziprasidone is a combination of ziprasidone and a solubilizing agent. Examples of solubilizing agents include surfactants; pH control agents such as buffers, organic acids (as examples, citric acid and gluconic acid); glycerides; partial glycerides; glyceride derivatives; polyoxyethylene and polyoxypropylene ethers and their copolymers; sorbitan esters; polyoxyethylene sorbitan esters; alkyl sulfonates; phospholipids; and lipophilic mircophase-forming materials as described in US published patent application 2003/0228358A1 which is incorporated herein by reference. Such solubilizing agents are known in the art, and any known such agent is contemplated for use in the present invention.

The ziprasidone formulation in the methods, kits and dosage forms of the present invention may usefully comprise one or more precipitation inhibitors. Accordingly, it may be useful to combine the dissolution rate-improved form of ziprasidone or the solubility-improved form of ziprasidone with one or more precipitation inhibitors. By a “precipitation inhibitor” is meant any material known in the art that is capable of slowing the rate at which ziprasidone crystallizes or precipitates from an aqueous solution that is supersaturated with ziprasidone. Precipitation inhibitors suitable for use in the dosage forms of the present invention should be inert, in the sense that they do not chemically react with ziprasidone in an adverse manner, be pharmaceutically acceptable, and have at least some solubility in aqueous solution at physiologically relevant pHs (e.g. 1-8). The precipitation inhibitor can be neutral or ionizable, and should have an aqueous-solubility of at least 0.1 mg/mL over at least a portion of the pH range of 1-8.

Precipitation inhibitors may be polymers or non-polymeric. Precipitation-inhibiting polymers suitable for use with the present invention may be cellulosic or non-cellulosic. The polymers may be neutral (i.e. substantially non-ionizable in aqueous solution) or ionizable in aqueous solution. For example, precipitation inhibitors used in the present invention may be selected from neutral non-cellulosic polymers, ionizable non-cellulosic polymers, neutral cellulosic polymers, and ionizable cellulosic polymers. Of these, ionizable and cellulosic polymers are preferred, with ionizable cellulosic polymers being more preferred. Also preferred are polymers that are “amphiphilic” in nature, meaning that the polymer has hydrophobic and hydrophilic portions. Precipitation inhibitors that may be useful in the present invention are well known in the art and are described, for example, in U.S. published patent application 2006/0003011A1 and in U.S. published patent application 2007/0190129, both of which are incorporated herein in their entireties by reference.

Exemplary cellulosic polymers that are at least partially ionized at physiologically relevant pHs include: hydroxypropyl methyl cellulose acetate succinate (HPMCAS), hydroxypropyl methyl cellulose phthalate (HPMCP), carboxymethyl ethyl cellulose (CMEC), cellulose acetate phthalate (CAP), hydroxypropyl methyl cellulose acetate phthalate, cellulose acetate trimellitate (CAT), cellulose acetate terephthalate, and cellulose acetate isophthalate.

Exemplary non-ionizable cellulosic polymers include hydroxypropyl methyl cellulose acetate (HPMCA), hydroxypropyl methyl cellulose (HPMC), hydroxypropyl cellulose, methyl cellulose, hydroxyethyl methyl cellulose, hydroxyethyl cellulose acetate, and hydroxyethyl ethyl cellulose.

Exemplary non-cellulosic polymers include vinyl polymers and copolymers having substituents of hydroxyl, alkylacyloxy, or cyclicamido; polyvinyl alcohols that have at least a portion of their repeat units in the unhydrolyzed (vinyl acetate) form; polyvinyl alcohol polyvinyl acetate copolymers; polyvinyl pyrrolidone; polyoxyethylene-polyoxypropylene copolymers, also known as poloxamers; polyethylene polyvinyl alcohol copolymers; carboxylic acid-functionalized vinyl polymers, such as the carboxylic acid functionalized polymethacrylates and carboxylic acid functionalized polyacrylates such as the EUDRAGITS® manufactured by Rohm Tech Inc., of Malden, Mass.; amine-functionalized polyacrylates and polymethacrylates; proteins; and carboxylic acid functionalized starches such as starch glycolate.

Preferred precipitation inhibitors include hydroxypropyl methyl cellulose acetate succinate, hydroxypropyl methyl cellulose phthalate, carboxymethyl ethyl cellulose, cellulose acetate phthalate, cellulose acetate trimellitate, hydroxypropyl methyl cellulose, polyvinyl pyrrolidone, polyoxyethylene-polyoxypropylene copolymers, and mixtures thereof.

The combination of ziprasidone and precipitation inhibitor may be prepared by methods known in the art such as dry- or wet-mixing the drug or drug mixture with the precipitation inhibitor to form the composition. Mixing processes include physical processing as well as wet-granulation and coating processes.

Milling may also be employed to prepare the compositions of the present invention. The milling process may serve simultaneously as a mixing process if the feed materials are heterogeneous. Conventional mixing and milling processes suitable for use in the present invention are discussed more fully in Lachman, et al., The Theory and Practice of Industrial Pharmacy (3rd Ed. 1986). The components of the compositions may also be combined by dry- or wet-granulating processes.

In one embodiment, the combination comprises particles of the ziprasidone coated, for example by a spray drying process, with a precipitation-inhibiting polymer. The particles may be either ziprasidone crystals, or particles of some other form of ziprasidone such as amorphous drug or a cyclodextrin complex.

The amount of precipitation inhibitor may vary widely. The weight ratio of ziprasidone to precipitation inhibitor may range from 100 to 0.01. Where the precipitation inhibitor is a polymer, preferably the polymer to drug weight ratio is at least 0.33 (at least 25 wt % polymer), more preferably at least 0.66 (at least 40 wt % polymer), and even more preferably at least 1 (at least 50 wt % polymer).

### Solid Oral Dosage Forms:

The solid oral dosage forms of the present invention may be in any of those forms known in the art, for example hard or soft capsules, sachets, lozenges, or tablets, in accordance with the present invention. In another embodiment, the oral administration may be in a powder, bead, multiparticulate, or granule form, such as in sachets. In another embodiment, the oral dose form is sub-lingual, such as, for example, a lozenge. The solid oral dosage forms of the invention may contain a sustained release means, a delayed release means or a slow-disintegrating portion. In the case of capsules and tablets the dosage forms also may comprise buffering agents or may be prepared with enteric coatings.

The effective daily amount for ziprasidone when administered orally is in general from about 10 mgA to 200 mgA per day. The total daily amount of ziprasidone can be adjusted by a physician of ordinary skill in the art, taking into account relevant factors as is known to those of ordinary skill in the art, for example the patient's weight or the severity of the CNS affliction. This total daily amount of ziprasidone may be given in a single or divided doses. Preferably, the total daily amount is from 40 mgA to 160 mgA and is given in two doses per day. Accordingly, a unit of a solid oral dosage form of the present invention preferably contains between 20 mgA ziprasidone and 80 mgA ziprasidone. Preferably a solid oral dosage form according to the methods of the present invention contains 20, 40, 60 or 80 mgA ziprasidone. In another embodiment, the solid oral dosage form of the present invention contains 120 mgA or 160 mgA ziprasidone.

In addition to the active ingredient, solid oral dosage forms according to this invention may be formulated to optionally include a variety of one or more conventional excipients, depending on the exact formulation, such as disintegrants, binders, flavorings, buffers, diluents, colors, lubricants, sweetening agents, thickening agents, and glidants. Some excipients can serve multiple functions, for example as both binder and disintegrant.

Generally, excipients such as surfactants, pH modifiers, fillers, matrix materials, complexing agents, solubilizers, pigments, lubricants, glidants, flavorants, and so forth may be used for customary purposes and in typical amounts without adversely affecting the properties of the sustained release dosage form. See for example, Remington's Pharmaceutical Sciences (18th ed. 1990).

Conventional matrix materials, complexing agents, solubilizers, disintegrating agents (disintegrants), or binders may also comprise up to 90 wt % of the dosage form.

Examples of fillers, or diluents include lactose, mannitol, xylitol, microcrystalline cellulose, dibasic calcium phosphate (anhydrous and dihydrate) and starch.

Examples of disintegrants include sodium starch glycolate, sodium alginate, carboxy methyl cellulose sodium, methyl cellulose, and croscarmellose sodium, and crosslinked forms of polyvinyl pyrrolidone such as those sold under the trade name CROSPOVIDONE (available from BASF Corporation).

Examples of binders include methyl cellulose, microcrystalline cellulose, starch, and gums such as guar gum, and tragacanth.

Examples of lubricants include magnesium stearate, calcium stearate, and stearic acid.

Examples of preservatives include sulfites (an antioxidant), butyrated hydroxytoluene, butyrated hydroxyanisole, benzalkonium chloride, methyl paraben, propyl paraben, benzyl alcohol and sodium benzoate.

Examples of anti-caking agents or fillers include silicon oxide and lactose.

Other conventional excipients may be employed in the sustained release dosage forms of this invention, including those well-known in the art. Generally, excipients such as pigments, lubricants, flavorants, and so forth may be used for customary purposes and in typical amounts without adversely affecting the properties of the compositions.

### Sustained Release Means and Delayed Release Means

The Cmax of a dosage form comprising a dissolution rate improved or solubility-improved form of ziprasidone can be higher than the Cmax of a control immediate relapse oral capsule dosed in the fed state. To attenuate this Cmax, the dosage form can be modified using a sustained release or delayed release dosage form comprising the dissolution rate improved or solubility-improved form of ziprasidone, either alone or in combination with an immediate release portion of ziprasidone. Sustained release formulations comprising ziprasidone can result in the lowering of the observed serum Cmax in mammals relative to oral immediate release dosage forms of the same ziprasidone dose. The sustained release dosage forms of the present invention resulting in a lower Cmax possess a slower rate of drug release from the dosage form. In this embodiment, the ziprasidone released from the dosage form is preferably in the form of the dissolution rate-improved or solubility-improved form, or both. Preferably, a sustained release means in a dosage form of the present invention releases no more than 50% of the ziprasidone therein (within the sustained release means) within ½ hour, more preferably no more than 50% within 1 hour, subsequent to introduction of the dosage form containing the sustained release means to a test media containing 900 ml (0.05M Na₂HPO₄, 2% SDS pH 7.5) at 37 C, with stirring at 100 RPM.

In various embodiments, the dosage forms, methods and kits of the subject invention comprise a sustained release means. Such components, when incorporated into the solid oral ziprasidone dosage form, can be any of those known to a person of ordinary skill in the art. Exemplary dosage forms include erodible and non-erodible matrix sustained-release dosage forms, osmotic sustained-release dosage forms, multiparticulates, and dosage forms comprising an enteric coated core. Such dosage forms are described, for example, in U.S. published patent application 2007/0190129, supra.

In one embodiment, the dosage form is an erodible or non-erodible polymeric matrix sustained release dosage form. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous use environment, the erodible polymeric matrix imbibes water and forms an aqueous-swollen gel or “matrix” that entraps the ziprasidone. The aqueous-swollen matrix gradually erodes, swells, disintegrates, disperses or dissolves in the environment of use, thereby controlling the release of ziprasidone to the environment of use. Examples of such dosage forms are well known in the art. See, for example, Remington The Science and Practice of Pharmacy, 20^{th} Edition, 2000.

A key ingredient of the water-swollen matrix is the water-swellable, erodible, or soluble polymer, which may generally be described as an osmopolymer, hydrogel or water-swellable polymer. Such polymers may be linear, branched, or crosslinked. They may be homopolymers or copolymers. Exemplary polymers include naturally occurring polysaccharides such as chitin, chitosan, dextran and, pullulan; gum agar, gum arabic, gum karaya, locust bean gum, gum tragacanth, carrageenans, gum ghatti, guar gum, xanthan gum and scleroglucan; starches such as dextrin and maltodextrin; hydrophilic colloids such as pectin; phosphatides such as lecithin; alginates such as ammonium alginate, sodium, potassium or calcium alginate, propylene glycol alginate; gelatin; collagen; and cellulosics. By “cellulosics” is meant a cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent.

A preferred class of cellulosics for the erodible matrix comprises aqueous-soluble and aqueous-erodible cellulosics such as ethyl cellulose (EC), methylethyl cellulose (MEC), carboxymethyl cellulose (CMC), carboxymethyl ethylcellulose (CMEC), hydroxyethyl cellulose (HEC), hydroxypropyl cellulose (HPC), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC).

A particularly preferred class of such cellulosics comprises various grades of low viscosity (MW less than or equal to 50,000 daltons) and high viscosity (MW greater than 50,000 daltons) HPMC. Commercially available low viscosity HPMC polymers include the Dow METHOCEL™ series E3, E5, E15LV, E50LV and K100LV, while high viscosity HPMC polymers include E4MCR, E10MCR, K4M, K15M and K100M; especially preferred in this group are the METHOCEL™ K series. Other commercially available types of HPMC include the Shin Etsu METOLOSE™ 90SH series. In one embodiment, the HPMC has a low viscosity, meaning that the viscosity of a 2% (w/v) solution of the HPMC in water is less than about 120 cp. A preferred HPMC is one in which the viscosity of a 2% (w/v) solution of the HPMC in water ranges from 80 to 120 cp (such as METHOCEL™ K100LV).

Other materials useful as the erodible matrix material include, but are not limited—to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl) methacrylate chloride.

The erodible matrix polymer may also contain additives and excipients known in the pharmaceutical arts, including osmopolymers, osmagens, solubility-enhancing or retarding agents and excipients that promote stability or processing of the dosage form.

Alternatively, the sustained-release portion may comprise a non-erodible matrix. In such dosage forms, the ziprasidone is distributed in an inert matrix. The drug is released by diffusion through the inert matrix. Examples of materials suitable for the inert matrix include insoluble plastics, such as copolymers of ethylene and vinyl acetate, methyl acrylate-methyl methacrylate copolymers, polyvinyl chloride, and polyethylene; hydrophilic polymers, such as ethyl cellulose, cellulose acetate, and crosslinked polyvinylpyrrolidone (also known as crospovidone); and fatty compounds, such as carnauba wax, microcrystalline wax, and triglycerides. Such dosage forms are described further in Remington: The Science and Practice of Pharmacy, 20th edition (2000).

Thus, in one embodiment, the sustained release dosage form comprises 10 wt % to 80 wt % of ziprasidone in a dissolution rate-improved or solubility-improved form, 5 wt % to 50 wt % of a matrix polymer, and 10 wt % to 85 wt % of a diluent. In one embodiment, the ziprasidone is in the form of a solid dispersion of semi-ordered ziprasidone and a polymer. In another embodiment, the ziprasidone is in the form of a solid dispersion of semi-ordered ziprasidone hydrochloride and HPMCAS. In another embodiment, the ziprasidone is in the form of a solid dispersion of semi-ordered ziprasidone free base and HPMC. In another embodiment, the ziprasidone is in the form of a solid dispersion of ziprasidone mesylate and SBECD. In one embodiment, the matrix polymer is HPMC. In another embodiment, the diluent is lactose monohydrate.

In another embodiment, the dosage form is an osmotic dosage form. In osmotic delivery devices, an osmotic agent (a water-swellable hydrophilic polymer or an osmogen or osmagent) is included in the device core, and the core is coated with a semipermeable membrane. The membrane may or may not include one or more delivery ports formed during membrane formation, following the coating process, or in situ. Delivery ports may range from a single port to many small delivery ports that may consist of pores in the coating. The osmotic agent inside the core draws water through the semipermeable coating. For cores containing a water-swellable hydrophilic polymer, the core imbibes water through the coating, swelling the water-swellable composition and increasing the pressure within the core, and fluidizing the drug-containing composition. Because the coating remains intact, the drug-containing composition is extruded out through the one or more delivery ports or pores in the coating into an environment of use. For cores containing an osmogen, water is osmotically drawn into the device and dissolves the agent, forming a solution of the agent. The increase in volume caused by the imbibition of water raises the hydrostatic pressure inside the core slightly. This pressure is relieved by a flow of saturated agent solution or suspension out of the device through the membrane pores or a delivery port. Thus, the volume-flow rate from devices containing water-swellable polymers or osmogens is dependent on the rate of water influx through the membrane to the core and the product of the drug concentration in the extruded fluid. Porous, asymmetric, symmetric, or phase inversion membranes may be used to control the rate of water influx and, in turn, the rate of drug release for osmotic controlled release devices.

A dosage form according to the present invention having a sustained release component may optionally also have an immediate release portion which comprises ziprasidone. By “immediate release portion” is meant broadly that a portion of the ziprasidone separate from the sustained release component is released within the first two hours or less following administration to a gastric use environment. Immediate release of drug may be accomplished by any means known in the pharmaceutical arts, including immediate release coatings, immediate release layers, and immediate release multiparticulates or granules. Sustained release dosage forms comprising an immediate release portion are described in the art, for example in U.S. published patent, application 2007/0190129, supra.

A dosage form useful in the present invention may also comprise a delayed release means, either alone or in combination with a sustained release means and/or immediate release portion. Examples of delayed release means which may be used in the sustained release ziprasidone dosage forms of the methods and kits of the present invention include but are not limited to dosage forms which comprise an enteric coated portion, delaying the release of the ziprasidone therein.

Some general examples of dosage forms comprising different combinations of immediate release, sustained release and/or delayed release components are as follows (these descriptions are not intended to limit the scope of dosage forms contemplated for use in the present invention):

### Immediate Release Core with Delayed Release Coating

In principle, the invention can be implemented by taking an immediate release core comprising ziprasidone and a pharmaceutically acceptable carrier and coating it with a (preferably all-covering) coating which provides the desired delayed release characteristics, either by a spatial or temporal mechanism. Thus any immediate release ziprasidone dosage form can be used as a core which is in turn coated with a desired delayed-release coating, and such dosage forms constitute preferred embodiments within the scope of this invention.

The dosage form can operate by being sensitive to its use environment such that it delays releasing ziprasidone until after it has passed into the small intestine. This type of delayed release dosage form releases in a manner which is dependent on position along the gastrointestinal (GI) tract, is independent of time, and is herein referred to as a “spatial” dosage form, or as exhibiting “spatially delayed release”. After the dosage form has entered the small intestine, it releases its remaining ziprasidone in immediate fashion, “immediate release” meaning that no component or means is implemented in the dosage form which would deliberately retard or slow down release once the delay period has ended: In general, the dosage form should release at least 70% of the ziprasidone remaining therein within 1.5 hours, preferably within one hour, after passing into the small intestine. An examples of spatially delayed dosage form is pH-triggered dosage forms which delay release of ziprasidone until they enter the environment of the small intestine, which is above pH 5.5 Spatially-delayed dosage forms of this invention generally commence immediate release of ziprasidone within approximately 30 minutes, preferably within 15 minutes, after passing out of the stomach into the small intestine.

### Immediate Release with Spatially Delayed pH-Triggered Coating

A first spatially-delayed release embodiment according to the invention is a “pH-dependent coated tablet”, which comprises an immediate-release tablet or tablet core coated with a material comprising a polymer that is substantially impermeable to ziprasidone at the pH of the stomach, but which becomes permeable to ziprasidone at the pH of the small intestine. “Substantially impermeable” in relation to spatially delayed dosage forms allows for very small amounts of ziprasidone to be released through the coating, so long as not more than 10% of the ziprasidone contained in the dosage form is released in the stomach. Such polymers become permeable by virtue of dissolving or disintegrating or otherwise being disrupted so that ziprasidone can freely pass through. The tablet or tablet core can comprise further excipients such as disintegrants, lubricants, fillers, and/or other conventional formulation ingredients. All such ingredients and/or excipients, regardless of the particular dosage form, are referred to herein collectively as the pharmaceutically acceptable “carrier”. The core is coated with a material, preferably a polymer, which is substantially insoluble and impermeable at the pH of the stomach, but which is more permeable at the pH of the small intestine. Preferably, the coating polymer is substantially insoluble and impermeable at pH <5.0, and water-soluble or water-disintegrable at pH>5.0. Mixtures of a pH-sensitive polymer with a water-insoluble polymer may also be employed. Tablets are coated with an amount of polymer comprising from 3% to 70% of the weight of the ziprasidone-containing tablet core. Preferred tablets are coated with an amount of polymer comprising 5% to 50% of the weight of the ziprasidone-containing tablet core.

pH-sensitive polymers which are relatively insoluble and impermeable at the pH of the stomach, but which are more soluble or disintegrable or permeable at the pH of the small intestine and colon include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, hydroxypropyl methylcellulose acetate succinate, cellulose acetate succinate, cellulose acetate trimellitate, styrene-maleic acid dibutyl phthalate copolymer, styrene-maleic acid polyvinylacetate phthalate copolymer, styrene and maleic acid copolymers, polyacrylic add derivatives such as acrylic acid and acrylic ester copolymers, polymethacrylic acid and esters thereof, poly acrylic methacrylic add copolymers, shellac, and vinyl acetate and crotonic add copolymers.

Preferred pH-sensitive polymers include shellac, phthalate derivatives, particularly cellulose acetate phthalate, polyvinylacetate phthalate, and hydroxypropylmethylcellulose phthalate; hydroxypropyl methylcellulose acetate succinate; cellulose acetate trimellitate; polyacrylic acid derivatives, particularly copolymers comprising acrylic acid and at least one acrylic, acid ester, polymethyl methacrylate blended with acrylic add and acrylic ester copolymers; and vinyl acetate and crotonic add copolymers.

A particularly preferred group of pH-sensitive polymers includes cellulose acetate phthalate, polyvinylacetate phthalate, hydroxypropylmethylcellulose phthalate, hydroxypropyl methylcellulose acetate succinate, anionic acrylic copolymers of methacrylic acid and methylmethacrylate, and copolymers comprising acrylic acid and at least one acrylic add ester.

Cellulose acetate phthalate (CAP) may be applied to ziprasidone dosage forms to provide delayed release of ziprasidone until the ziprasidone-containing tablet has exited the stomach. The CAP coating solution may also contain one or more plasticizers, such as diethyl phthalate, polyethyleneglycol-400, triacetin, triacetin citrate, propylene glycol, and others as known in the art. Preferred plasticizers are diethyl phthalate and triacetin. The CAP coating formulation may also contain one or more emulsifiers, such as polysorbate-80.

Anionic acrylic copolymers of methacrylic acid and methylmethacrylate are also particularly useful coating materials for delaying the release of ziprasidone from ziprasidone-containing tablets until the tablets have moved to a position in the GI tract which is distal to the stomach. Copolymers of this type are available from RoehmPharma Corp, under the trademarks Eudragit®-L and Eudragit®-S. Eudragit®-L and Eudragit®-S are anionic copolymers of methacrylic acid and methylmethacrylate. The ratio of free carboxyl groups to the esters is approximately 1:1 in Eudragit®-L and approximately 1:2 in Eudragit®-S. Mixtures of Eudragit®-L and Eudragit®-S may also be used. For coating of ziprasidone-containing tablets, these acrylic coating polymers can be dissolved in an organic solvent or mixture of organic solvents or suspended in aqueous media. Useful solvents for this purpose are acetone, isopropyl alcohol, and methylene chloride. It is generally advisable to include 5-20% plasticizer in coating formulations of acrylic copolymers. Useful plasticizers include polyethylene glycols, propylene glycols, diethyl phthalate, dibutyl phthalate, castor oil, triethyl citrate, and triacetin. Eudragit®-L is preferred because it dissolves relatively quickly at intestinal pH.

A coating of hydroxypropyl methylcellulose acetate succinate may also be applied to the dosage forms to provide delayed release of ziprasidone until the dosage form has exited the stomach.

The coating, as noted above, may comprise from 3% to 70% of the weight of the uncoated tablet core. Preferably, the coating comprises from 5% to 50%, more preferably 5% to 40% of the weight of the tablet core.

In a further embodiment of a spatially-delayed ziprasidone dosage form, a “pH-dependent coated bead”, beads 0.4 to 2.0 mm in diameter comprising ziprasidone plus carrier are coated with one or more of the aforementioned pH-sensitive polymers. The coated beads may be placed in a capsule or may be compressed into a tablet, with care taken to avoid damaging the polymeric coat on individual beads during tablet compression. Preferred coated beads are those which exhibit essentially no release (i.e., less than 10%) of ziprasidone from the dosage form, as previously discussed, until the beads have exited the stomach, thus assuring that minimal ziprasidone is released in the stomach. The coating may comprise from 5% to 200% of the weight of the uncoated bead core. Preferably, the coating comprises from 10% to 100% of the weight of the bead core.

In a further embodiment of a multiparticulate spatially-delayed ziprasidone dosage form, a “pH-dependent coated particle”, the dosage form comprises small ziprasidone plus carrier particles from 0.1 to 0.4 mm in diameter. The particles are coated with one or more of the aforementioned pH-sensitive polymers. The coated particles may be used to make unit dose packs or may be placed in a capsule or may be compressed into a tablet, with care taken to avoid damaging the polymeric coat on individual particles during tablet compression.

In still another embodiment, the multiparticulate spatially-delayed ziprasidone dosage form comprises enteric coated granules comprising ziprasidone, granulation excipients, and an enteric polymer. These granules have diameters from 0.1 to 1 mm, and can be made using techniques known in the art. In one embodiment, ziprasidone in dissolution rate-improved or solubility-improved form is mixed with a binder and an enteric polymer, and granulated using conventional granulation equipment.

Preferred coated particles are those which exhibit essentially no release of ziprasidone from the dosage form (i.e. less than 10%) until the particles have exited the stomach, thus assuring that minimal ziprasidone is released in the stomach. Mixtures of a pH-sensitive polymer with a water-insoluble polymer are also included. Preferred ziprasidone-containing particles are coated with an amount of polymer comprising 15% to 200% of the weight of the uncoated ziprasidone-containing particle core.

Mixtures of a pH-sensitive polymer with a water-insoluble polymer are also included. Ziprasidone-containing tablets and particles and beads may be coated with mixtures of polymers whose solubilities vary at different pHs. For example, preferred coatings comprise Eudragit®-L, or from 9:1 to 1:4 Eudragit®-L/Eudragit®-S.

A further embodiment of a spatially-delayed ziprasidone dosage form constitutes a modification of the pH-dependent coated tablet, pH-dependent coated bead, and pH-dependent coated particle embodiments. The ziprasidone-containing core tablet, bead, or particle is first coated with a barrier coat, and then is coated with the pH-dependent coat The function of the barrier coat is to separate ziprasidone from the pH-dependent coat. Since ziprasidone is a base, hydration of the ziprasidone in the core can serve to raise the pH in the microenvironment of the pH-dependent coating, thus prematurely initiating the permeabilization or dissolution of the pH-dependent coating, resulting in premature release of some or all of the ziprasidone dose in the stomach. A barrier coat prevents such premature release. Suitable barrier coatings are composed of water-soluble materials such as sugars such as sucrose, or water-soluble polymers such as hydroxypropyl cellulose, hydroxypropyl methylcellulose, and the like. Hydroxypropyl cellulose and hydroxypropylmethylcellulose and polyvinylpyrrolidone are preferred. The barrier coat may comprise from 1% to 20%, preferably from 2% to 15%, of the weight of the uncoated ziprasidone-containing tablet, bead or particle core.

### Delayed-Release Immediate Release Core with Immediate Release Coating

An example of another combination contemplated as useful for a ziprasidone dosage form for use in the present invention is a solid oral dosage form comprising a core providing for the immediate release of ziprasidone, which core is coated by a material to delay the release of the contents of the core (a “delayed-release coating”), as explained above. This delayed-release core for immediate release of ziprasidone is in turn coated with a layer comprising ziprasidone providing for the immediate release of ziprasidone.

Kits:

The subject invention also provides kits for treatment of a CNS disorder in a human comprising a solid oral dosage form as described herein comprising an effective amount of ziprasidone, which solid oral dosage form provides serum ziprasidone concentration levels effective for treating a CNS disorder, even when the human is in a fasted state. Accordingly, the kit also includes instructions for administration which do not specify administration with food.

In one embodiment, the subject invention provides a kit comprising
a) a solid oral dosage form comprising an effective amount of ziprasidone and a pharmaceutically acceptable carrier; and
b) instructions for oral administration of the dosage form of (a), which do not specify administration with food;

wherein said solid oral dosage form when administered to a human in a fasted state provides to the human a serum ziprasidone AUC_{0-inf} which is from 70% to 140%, preferably from 75% to 130%, more preferably from 80% to 125%, of the ziprasidone serum AUC_{0-inf} resulting from administration to a human in a fed state of a control ziprasidone immediate release oral capsule containing the same amount of ziprasidone.

In another embodiment, the subject invention provides a kit comprising
a) a solid oral dosage form comprising an effective amount of ziprasidone and a pharmaceutically acceptable carrier; and
b) instructions for oral administration of the dosage form of (a), which instructions indicate that the dosage form of (a) may be administered with or without food;

wherein said solid oral dosage form when administered to a human in a fasted state provides to the human a serum ziprasidone AUC_{0-inf} which is from 70% to 140%, preferably from 75% to 130%, more preferably from 80% to 125%, of the ziprasidone serum AUC_{0-inf} resulting from administration to a human in a fed state of a control ziprasidone immediate release oral capsule containing the same amount of ziprasidone.

In another embodiment, the subject invention provides a kit comprising
a) a solid oral dosage form comprising an effective amount of ziprasidone and a pharmaceutically acceptable carrier; and
b) instructions for oral administration of the dosage form of (a), which do not specify administration with food;

wherein said solid oral dosage form when administered to a human in a fasted state provides to the human a serum ziprasidone AUC_{0-inf} which is from 70% to 140%, preferably from 75% to 130%, more preferably from 80% to 125%, of the ziprasidone serum AUC_{0-inf} resulting from administration of an identical ziprasidone solid oral dosage form to a human in a fed state.

In another embodiment, the subject invention provides a kit comprising
a) a solid oral dosage form comprising an effective amount of ziprasidone and a pharmaceutically acceptable carrier; and
b) instructions for oral administration of the dosage form of (a), which instructions indicate that the dosage form of (a) may be administered with or without food;

wherein said solid oral dosage form when administered to a human in a fasted state provides, to the human a serum ziprasidone AUC_{0-inf} which is from 70% to 140%, preferably from 75% to 130%, more preferably from 80% to 125%, of the ziprasidone serum AUC_{0-inf} resulting from administration of an identical ziprasidone solid oral dosage form to a human in a fed state.

Preferably, the ziprasidone in the solid oral dosage form in a kit of the present invention is a dissolution rate-improved form of ziprasidone and/or a solubility-improved form of ziprasidone, as discussed above.

The term “kit” herein refers to any combination of solid oral dosage form and instructions. The instructions may be in a recorded form. The recording may for example be hand-written, printed, a video-recording or an audio-recording. The instructions may be part of a label, package insert, or other recorded information approved by a government regulatory agency for accompanying the distribution of the drug to consumers. The combination may be such that the solid oral dosage form and the instructions, in for example printed or written form, are part of the same package provided by a pharmacy to physicians, health administrators or patients.

The kit thus can be suitable for commercial sale, and can comprise a container, the solid oral dosage form of ziprasidone as described herein, and associated written (e.g., printed) matter non-limited as to whether the dosage form can be taken with or without food. The written matter is of the type containing information and/or instructions for the physician, pharmacist or patient. The written material can be “non-limited as to whether the dosage form can be taken with or without food” by virtue of including no statement regarding whether or not the dosage form can be taken with or without food, i.e. the statement is silent with regard to food effects. Alternatively, the written material can be non-limited by containing one or more statements affirmatively informing the user (e.g., the patient, pharmacist, or physician) that the said oral dosage form can be taken by or administered to a patient regardless of whether the patient has eaten or otherwise imbibed food (optionally, for example, also stating something like “without regard to type or quantity of food”). The written material can not contain language requiring that the ziprasidone dosage form of the present invention must be administered to a subject in a fed state or with at least or more than 250 calories. In other words, the instructions, e.g. written material, cannot state “This ziprasidone dosage form must be taken with food” or “This ziprasidone dosage form may only be given after the patient has consumed a meal or snack” or the like.

The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual dosages for pressing out of the pack according to a therapeutic schedule. It is feasible that more than one container, can be used together in a single package to market a single dosage form. For example, tablets may be contained in a bottle which is in turn contained within a box.

Instructions, for example printed or otherwise written matter, is associated with the package in which the ziprasidone dosage form is sold. The term “associated with” is intended to include all manners in which instructions can be associated with a medicament. For example, written matter can be associated with the container by being: written on a label (e.g., the prescription label or a separate label), adhesively affixed to a bottle containing a ziprasidone dosage form; included inside a container as a written package insert, such as inside a box which contains unit dose packets; applied directly to the container such as being printed on the wall of a box; or attached as by being tied or taped, for example as an instructional card affixed to the neck of a bottle via a string, cord or other line, lanyard or tether type device. The written matter may be printed directly on a unit dose pack or blister pack or blister card.

Another example in which instructions may be “associated with” the ziprasidone solid oral dosage form is electronically. The instructions may be provided for example by a manufacturer on a web site offered by the manufacturer to consumers in connection with the ziprasidone solid oral dosage form.

The following examples illustrate the present invention. Additional embodiments of the present invention may be prepared using information presented in these examples, either alone or in combination with techniques generally known in the art. These examples are provided for illustration of the invention and therefore should not be construed to limit the intended scope of the invention, as described fully in the entire specification and claims of this application.

### EXAMPLES

Of the following Examples relating to clinical studies, each study was a randomized, open-label, 3-period, 6-sequence crossover study to examine the bioavailability of the GEODON/ZELDOX™ commercial capsule under fed conditions and of a test ziprasidone dosage form under fed and fasting conditions, all after single 40 mg doses.

All subjects were healthy and at least 18 years of age, with a Body Mass Index (BMI) of approximately 18 to 30 kg/m² and a total body weight >50 kg (110 lb).

Subjects were randomized to 1 of 6 treatment sequences on Day 1 of Period 1. The 3 treatments administered were test ziprasidone dosage form (Fasted), test ziprasidone dosage form (Fed) and Commercial Capsule (Fed). Each treatment period was separated by a minimum 3-day washout interval.

For the test formulation (Fasted) treatment, subjects were administered the drug with 240 mL (8 fluid oz) of water following an overnight fast of at least 10 hours. In case of test formulation (Fed) and Commercial Capsule (Fed) treatments, subjects were provided with breakfast following an overnight fast of at least 10 hours. The breakfast was consumed over a 20-minute period with the drug administered within 5 minutes after completion of the meal with 240 mL (8 fluid oz) of water.

Pharmacokinetic Evaluations: Blood samples (5 mL) sufficient to provide a minimum of 2 mL of serum for pharmacokinetic (PK) analysis of ziprasidone were collected in appropriately labeled red top tubes (not containing any serum separator or other additives) at the following times: Hour 0 (before the morning dose on Day 1), and 1, 2, 4, 6, 8, 10, 12, 16, 24 and 36 hours after drug administration.

Serum samples were assayed for ziprasidone using validated liquid chromatography/dual mass spectrometry (LC/MS/MS) assay. The PK parameters were calculated for each subject by standard non-compartmental analysis of concentration-time data using WinNonlin, Version. 3.2. Maximum observed serum concentrations (Cₘₐₓ) and Tₘₐₓ (time of first occurrence of Cₘₐₓ) were observed directly from the experimental data (serum concentration versus time). The area under the serum concentration-time profile from time zero extrapolated to infinite time (AUC_{inf}) and area under the serum concentration-time profile from time zero to the time for the last quantifiable concentration (AUCₗₐₛₜ) were estimated by linear trapezoidal approximation.

The following PK parameters were summarized by formulation and fed state. For AUC_{inf} and Cₘₐₓ individual subject parameters were also plotted by formulation and fed state.

| Parameter | Summary statistics |
|---|---|
| AUC_{inf}, AUCₗₐₛₜ, Cₘₐₓ | N, arithmetic mean, median, coefficient of |
| | variation (CV %), standard deviation (SD), |
| | minimum, maximum, geometric mean |
| Tₘₐₓ | N, median, minimum, maximum |
| t_{1/2} | N, arithmetic mean, median, CV %, standard |
| | deviation (SD), minimum, maximum |

### Example 1

### Ziprasidone Solubilized with SBECD

Preparation of Ziprasidone Test Formulation: the Ziprasidone Test Formulation contained a ziprasidone mesylate-SBECD lyophilized powder combined with HPMCAS in the mass ratio of approximately 1:6:2. This formulation was prepared based on the procedure outlined below: The SBECD was charged to a flask containing Water for Injection (WFI) and heated until a solution was obtained. Ziprasidone mesylate was then charged to the flask and heated until a solution was obtained. The solution was cooled and held between 35 and 40 C before filtering through a 0.45 micron Kleenpak Ultipro N66 filter into a holding vessel. The solution was then transferred to trays. The solution was chilled to at least −40° C. before beginning the freeze dry cycle. Over a period of several weeks the temperature was increased. The final drying cycle reduced the moisture content to less than 2%. The lyophilized powder was then milled. 176.2 mg of the milled lyophilized powder (which was equivalent to 20 mg of ziprasidone) was mixed with 50 mg of HPMCAS (MF grade) and filled into a gelatin capsule.

Two ziprasidone capsules (test formulation) were dosed (2×20 mg) to obtain a 40 mg dose of ziprasidone.

The ziprasidone test formulation information is given in Table 1.

TABLE 1
Study Drug Information

| Study Drug | Potency | Formulation |
|---|---|---|
| Ziprasidone Mesylate/Sulfobutyl Ether β | 117.0 mg/g | API |
| Cyclodextrin Lyophilized powder | (anhydrous) | |
| HPMC Acetate Succinate AS/MF | 10 g | Excipient |

Results: A total of 16 subjects were assigned to treatment and 12 completed the study. Four subjects discontinued the study due to an unwillingness to participate. Three of these subjects received test formulation (Fed) and 1 received the Commercial Capsule (Fed) before discontinuing from the study.

The summary for PK parameters following single doses of Example 1 test formulation under fed and fasting state and commercial capsule under fed state are summarized in Table 2. These data show that the test formulation had a fasted AUC_{inf} that was 92% that of the fed AUC_{inf} of the test formulation, and the test formulation had a fasted AUC_{inf} that was 105% that of the commercial capsule fed.

TABLE 2
Summary for Ziprasidone Pharmacokinetic Parameters

| | Test Formulation | | Test Formulation | | Commercial | |
|---|---|---|---|---|---|---|
| | (Fasted) | | (Fed) | | Capsule (Fed) | |
| Parameter (Units) | N^{a} = 12 | | N^{a} = 13 | | N^{a} = 12 | |
| AUC_{inf} (ng · h/mL) | | | | | | |
| Geometric Mean (CV %) | 1195.6 | (17) | 1299.5 | (19) | 1137.3 | (17) |
| AUClast (ng · h/mL) | | | | | | |
| Geometric Mean (CV %) | 1187.4 | (17) | 1267.9 | (20) | 1127.3 | (17) |
| Cₘₐₓ (ng/mL) | | | | | | |
| Geometric Mean (CV %) | 179.6 | (29) | 161.4 | (21) | 118.8 | (23) |
| Tₘₐₓ (h) | | | | | | |
| Median (Range) | 2.0 | (1.0, 6.0) | 6.0 | (2.0, 8.0) | 6.0 | (4.0, 10.0) |
| t_{1/2} (h) | | | | | | |
| Arithmetic Mean (SD) | 4.95 | (0.624) | 4.44 | (0.866) | 4.59 | (0.600) |

CV = Coefficient of Variation;
SD = Standard Deviation
^{a}Number of subjects contributing to the summary statistics

### Example 2

### Ziprasidone Nanoparticles

Preparation of Candidate Formulation: this Test Formulation, Called Formulation B herein, contained ziprasidone free base in the form of nanoparticles. The formulation was prepared based on the procedure outlined below: A coarse suspension was prepared by placing 8.85 gm of ziprasidone free base in the 100 ml milling chamber with 48.89 gm of milling media (500 micron polystyrene beads). To this, 4.2 ml each of 10% solutions of Pluronic® F108, Tween® 80 and 5% Lecithin solutions were added. In addition, 23.8 ml of water for injection was added to the milling chamber. The above mixture was stirred until uniform suspension was obtained. This suspension was then milled for 30 minutes at 2100 RPM in a Nanomill-1 (Manufacturer Elan Drug Delivery, Inc.) and the temperature during the milling was maintained at 4 C. The resulting suspension was filtered under vacuum to remove the milling media. An appropriate volume of the suspension (corresponding to 40 mg dose of ziprasidone) was diluted in 60 ml water and dosed as a suspension.

The study drug information is given in Table 3.

TABLE 3
Study Drug Information

| Study Drug | Potency | Formulation |
|---|---|---|
| Ziprasidone Aqueous | 210 mg/ml | Suspension |
| Suspension 210 mg/mL Vial | | |

Results: A total of 14 subjects were assigned, to treatment and 12 completed the study. Two subjects (1 in Formulation B (Fasted) group and 1 in the Commercial Capsule (Fed) group) discontinued the study after Period 1 dosing. One subject discontinued due to gastroenteritis and the other was withdrawn by the sponsor.

The summary for PK parameters following single doses of Formulation B under fed and fasting state and Commercial capsule under fed state are summarized in Table 4. These data show that the test formulation had a fasted AUC_{inf} that was 78% that of the fed AUC_{inf} of the test formulation, and the test formulation had a fasted AUC_{inf} that was 89% that of the commercial capsule fed.

TABLE 4
Summary for Ziprasidone Pharmacokinetic Parameters

| | | | | | Commercial | |
|---|---|---|---|---|---|---|
| | Formulation B | | Formulation B | | Capsule (Fed) | |
| Parameter (Units) | (Fasted) N^{a} = 13 | | (Fed) N^{a} = 12 | | N^{a} = 12 | |
| AUC_{inf} (ng · h/mL) | | | | | | |
| Geometric Mean (CV %) | 962.4 | (42) | 1237.2 | (26) | 1084.9 | (31) |
| AUCₗₐₛₜ (ng · h/mL) | | | | | | |
| Geometric Mean (CV %) | 937.4 | (44) | 1224.1 | (26) | 1068.5 | (31) |
| Cₘₐₓ (ng/mL) | | | | | | |
| Geometric Mean (CV %) | 151.1 | (45) | 111.9 | (24) | 98.9 | (32) |
| Tₘₐₓ (h) | | | | | | |
| Median (Range) | 2.0 | (1.0, 4.0) | 4.0 | (1.0, 10.0) | 7.0 | (6.0, 16.0) |
| t_{1/2} (h) | | | | | | |
| Arithmetic Mean (SD) | 6.43 | (2.628) | 4.95 | (0.948) | 4.94 | (0.912) |

CV = Coefficient of Variation;
SD = Standard Deviation
^{a}Number of subjects contributing to the summary statistics

### Comparative Example 3

### Ziprasidone HCl

Ziprasidone coated crystals comprising 35% active ziprasidone hydrochloride monohydrate coated with the precipitation-inhibiting polymer HPMCAS, were prepared as described in U.S. published patent application 2007/0190129, supra.

This test ziprasidone formulation of Example 3 (called “Formulation C”) was dosed in as a powder-in-capsule (1×40 mg). The test drug information is given in Table 5.

TABLE 5
Study Drug Information

| Study Drug | Potency |
|---|---|
| Jet-milled ziprasidone HCl CCSD | 350 mg/g sent, Administered |
| coated crystals | 113.3 mg equivalent to 40 mg active |

Results: The summary for PK parameters following single doses of the test formulation under fed and fasting state and commercial capsule under fed state are summarized in Table 6. These data show that the test formulation had a fasted AUC_{inf} that was 57% that of the fed AUC_{inf} of the test formulation, and the test formulation had a fasted AUC_{inf} that was 62% that of the commercial capsule fed.

TABLE 6
Summary for Ziprasidone Pharmacokinetic Parameters

| | | | | | Commercial | |
|---|---|---|---|---|---|---|
| | Formulation C | | Formulation C | | Capsule | |
| Parameter (Units) | (Fasted) N^{a} = 12 | | (Fed) N^{a} = 15 | | (Fed) N^{a} = 12 | |
| AUC_{inf} (ng · h/mL) | | | | | | |
| Geometric Mean (CV %) | 822.3 | (33) | 1435.6 | (19) | 1320.6 | (20) |
| AUCₗₐₛₜ (ng · h/mL) | | | | | | |
| Geometric Mean (CV %) | 793.3 | (35) | 1418.3 | (19) | 1307.7 | (20) |
| Cₘₐₓ (ng/mL) | | | | | | |
| Geometric Mean (CV %) | 81.0 | (39) | 152.9 | (22) | 125.9 | (28) |
| Tₘₐₓ (h) | | | | | | |
| Median (Range) | 4.0 | (2.0, 10.0) | 8.0 | (6.0, 12.0) | 6.0 | (4.0, 12.0) |
| t_{1/2} (h) | | | | | | |
| Arithmetic Mean (SD) | 7.40 | (1.990) | 4.69 | (0.442) | 4.48 | (0.315) |

^{a}Number of subjects contributing to the summary statistics
CV = Coefficient of Variation;
SD = Standard Deviation

### Comparative Example 4

### Ziprasidone Mesylate, with a Precipitation Inhibitor (HPMCAS)

This formulation, called Formulation A2 herein, was a matrix tablet comprising hypromellose that contained ziprasidone mesylate in a simple physical mixture with a precipitation inhibitor, specifically HPMCAS. The composition of tablet of Formulation A2 was as follows:

| | Mass in |
|---|---|
| Component | Tablet (mg) |
| Ziprasidone Mesylate | 54.57 |
| Hydroxypropyl methylcellulose acetate succinate, MF grade | 218.28 |
| Croscarmellose sodium | 33.75 |
| Hypromellose (METHOCEL E5 Premium LV | 33.75 |
| Lactose monohydrate (FAST FLO 316) | 109.65 |
| TOTAL | 450.00 |

When tested by USP-2; 900 ml (0.05M Na2HPO4, 2% SDS pH 7.5) at 37 C and stirred at 100 RPM) the dissolution profile of Formulation A2 was

Formulation A2
Dissolution Profile (% Drug Dissolved)

| Time (hrs) | Tab #1 | Tab # 2 | Tab #3 | Average | Min | Max |
|---|---|---|---|---|---|---|
| 0.00 | 0.00 | 0.00 | 0.00 | 0.00 | 0.00 | 0.00 |
| 0.50 | 23.10 | 38.50 | 30.90 | 30.83 | 23.10 | 38.50 |
| 1.00 | 53.70 | 68.30 | 55.30 | 59.10 | 53.70 | 68.30 |
| 1.50 | 67.10 | 78.40 | 68.50 | 71.33 | 67.10 | 78.40 |
| 2.00 | 73.40 | 84.40 | 75.90 | 77.90 | 73.40 | 84.40 |
| 2.50 | 79.30 | 88.70 | 82.00 | 83.33 | 79.30 | 88.70 |
| 3.00 | 82.40 | 92.30 | 86.60 | 87.10 | 82.40 | 92.30 |
| 3.50 | 83.90 | 94.00 | 90.10 | 89.33 | 83.90 | 94.00 |
| 4.00 | 89.70 | 96.90 | 92.00 | 92.87 | 89.70 | 96.90 |
| 4.50 | 89.30 | 97.60 | 93.90 | 93.60 | 89.30 | 97.60 |
| 5.00 | 91.20 | 99.90 | 96.10 | 95.73 | 91.20 | 99.90 |
| 5.50 | 92.80 | 99.60 | 98.00 | 96.80 | 92.80 | 99.60 |
| 6.00 | 93.00 | 101.80 | 97.90 | 97.57 | 93.00 | 101.80 |

A total of 18 subjects were assigned to treatment with Formulation A2, and 17 subjects were assigned to the control GEODON/ZELDOX™ commercial capsules (Fed). 18 completed the study in Formulation A2 (Fasted) and 17 subjects each completed in Formulation A2 (Fed) and GEODON/ZELDOX™ commercial capsule (Fed).

Results: The mean serum PK parameters following single 40 mg doses of Formulation A2 under fasting or fed conditions and GEODON/ZELDOX™ commercial capsule under fed conditions are summarized in Table 7. These data show that the test formulation had a fasted AUC_{inf} that was 56% that of the fed AUC_{inf} of the test formulation, and the test formulation had a fasted AUC_{inf} that was 37% that of the commercial capsule fed.

TABLE 7
Mean Ziprasidone Pharmacokinetic Parameters

| | | Formulation | Geodon |
|---|---|---|---|
| | Formulation A2 | A2 (Fed) | Commercial |
| Parameter (units) | (Fasted) N = 18 | N = 18 | Capsule N = 17 |
| AUC_{inf} (ng · h/mL) | 339.4 | 608.2 | 909.7 |
| AUCₗₐₛₜ (ng · h/mL) | 329.0 | 598.4 | 900.0 |
| Cₘₐₓ (ng/mL) | 42.79 | 82.74 | 101.74 |
| Tₘₐₓ (h) | 4.0 | 4.0 | 6.0 |
| t_{1/2} (h) | 6.34 | 4.66 | 4.52 |

Geometric means for AUC_{inf}, AUCₗₐₛₜ and Cₘₐₓ; arithmetic mean for t_{1/2} and median for Tₘₐₓ.

### Example 5

### Ziprasidone Mesylate in Spray-Dried Dispersion with Cyclodextrin

Two formulations were prepared containing ziprasidone mesylate in a spray dried dispersion with the cyclodextrin sulfobutylether-beta-cyclodextrin (SBECD), as follows. First, a spray solution was prepared consisting of 7.8 wt % ziprasidone mesylate trihydrate, 30.9 wt % SBECD, all dissolved in water at 75° C. The feed solution was pumped to a spray drier (a Niro type XP Portable Spray-Dryer with a Liquid-Feed Process Vessel) (“PSD-1”), equipped with a pressure nozzle (Schlick 1.0 pressure nozzle). The PSD-1 was equipped with 9-inch chamber extension. The spray drier was also equipped with a DPH gas disperser for introduction of the drying gas to the spray drying chamber. The spray solution was pumped to the spray drier at about 54 g/min at a pressure of about 1000 psig. Drying gas (e.g., nitrogen) was introduced to the spray drier through the DPH lid at a flow rate of about 2000 g/min and at an inlet temperature of about 145° C. The evaporated water and drying gas exited the spray drier at a temperature of about 75° C. The resulting dispersion, containing 15% A ziprasidone, was collected in a cyclone.

The second formulation was prepared as described above with the following exceptions. The spray solution consisted of 6.1 wt % ziprasidone mesylate trihydrate, 31.8 wt % SBECD, all dissolved in water at 75° C. The spray solution was pumped to the spray drier at about 47 g/min at a pressure of about 800 psig. Drying gas (e.g., nitrogen) was introduced to the spray drier through the DPH lid at a flow rate of about 2000 g/min and at an inlet temperature of about 140° C. The evaporated water and drying gas exited the spray drier at a temperature of about 70° C. The resulting dispersion, containing 12% A ziprasidone, was collected in a cyclone.

Three solid oral dosage forms, called Formulation A3, Formulation A4, and Formulation A5, were tested. One of the formulations also included the precipitation inhibitor HPMCAS. The formulations are shown in Table 8.

TABLE 8
Composition of Formulations A3, A4, and A5

| | | Mass in |
|---|---|---|
| Formulation | Component | Tablet (mg) |
| A3 | 15% A Ziprasidone spray dried dispersion | 270.66 |
| | with SBECD | |
| | Lactose monohydrate (FAST FLO 316) | 69.34 |
| | Hypromellose (METHOCEL K100LV | 85.00 |
| | Premium CR) | |
| | Total | 425.00 |
| A4 | 15% A Ziprasidone spray dried dispersion | 270.66 |
| | with SBECD | |
| | Hydroxypropyl methylcellulose acetate | 109.14 |
| | succinate (MF grade) | |
| | Lactose monohydrate (FAST FLO 316) | 105.81 |
| | Hypromellose (METHOCEL K100LV | 39.38 |
| | Premium CR) | |
| | Total | 525.00 |
| A5 | 12% A Ziprasidone spray dried dispersion | 339.98 |
| | with SBECD | |
| | Hypromellose (METHOCEL K100LV | 95.89 |
| | Premium CR) | |
| | Total | 435.87 |

The dissolution profiles of formulations A3, A4, and A5 are shown below.

Formulation A3
Dissolution Profile (% Drug Dissolved)

| Time (hrs) | Tab #1 | Tab # 2 | Tab #3 | Average | Min | Max |
|---|---|---|---|---|---|---|
| 0.00 | 0.00 | 0.00 | 0.00 | 0.00 | 0.00 | 0.00 |
| 0.50 | 22.20 | 23.90 | 28.40 | 24.83 | 22.20 | 28.40 |
| 1.00 | 34.70 | 37.90 | 42.00 | 38.20 | 34.70 | 42.00 |
| 1.50 | 53.00 | 56.40 | 68.00 | 59.13 | 53.00 | 68.00 |
| 2.00 | 60.30 | 67.60 | 88.40 | 72.10 | 60.30 | 88.40 |
| 2.50 | 68.20 | 75.00 | 93.50 | 78.90 | 68.20 | 93.50 |
| 3.00 | 74.50 | 81.30 | 93.70 | 83.17 | 74.50 | 93.70 |
| 3.50 | 89.00 | 89.80 | 94.40 | 91.07 | 89.00 | 94.40 |
| 4.00 | 92.70 | 91.00 | 94.10 | 92.60 | 91.00 | 94.10 |
| 4.50 | 94.20 | 94.60 | 94.60 | 94.47 | 94.20 | 94.60 |
| 5.00 | 94.90 | 94.30 | 95.40 | 94.87 | 94.30 | 95.40 |
| 5.50 | 96.00 | 95.00 | 93.70 | 94.90 | 93.70 | 96.00 |
| 6.00 | 95.10 | 93.80 | 95.10 | 94.67 | 93.80 | 95.10 |

Formulation A4
Dissolution Profile (% Drug Dissolved)

| Time (hrs) | Tab #1 | Tab # 2 | Tab #3 | Average | Min | Max |
|---|---|---|---|---|---|---|
| 0.00 | 0.00 | 0.00 | 0.00 | 0.00 | 0.00 | 0.00 |
| 0.50 | 21.30 | 18.10 | 17.20 | 18.87 | 17.20 | 21.30 |
| 1.00 | 42.80 | 38.60 | 37.10 | 39.50 | 37.10 | 42.80 |
| 1.50 | 58.90 | 52.00 | 50.60 | 53.83 | 50.60 | 58.90 |
| 2.00 | 70.20 | 65.90 | 62.60 | 66.23 | 62.60 | 70.20 |
| 2.50 | 92.40 | 78.10 | 71.80 | 80.77 | 71.80 | 92.40 |
| 3.00 | 90.20 | 85.10 | 80.20 | 85.17 | 80.20 | 90.20 |
| 3.50 | 98.70 | 97.00 | 86.60 | 94.10 | 86.60 | 98.70 |
| 4.00 | 101.50 | 100.40 | 92.80 | 98.23 | 92.80 | 101.50 |
| 4.50 | 100.00 | 100.40 | 94.10 | 98.17 | 94.10 | 100.40 |
| 5.00 | 101.60 | 101.30 | 95.30 | 99.40 | 95.30 | 101.60 |
| 5.50 | 100.40 | 99.60 | 95.60 | 98.53 | 95.60 | 100.40 |
| 6.00 | 101.30 | 100.90 | 95.90 | 99.37 | 95.90 | 101.30 |

Formulation A5
Dissolution Profile (% Drug Dissolved)

| Time (hrs) | Tab #1 | Tab # 2 | Tab #3 | Average | Min | Max |
|---|---|---|---|---|---|---|
| 0.00 | 0.00 | 0.00 | 0.00 | 0.00 | 0.00 | 0.00 |
| 0.50 | 21.20 | 27.40 | 22.70 | 23.77 | 21.20 | 27.40 |
| 1.00 | 38.90 | 46.10 | 41.20 | 42.07 | 38.90 | 46.10 |
| 1.50 | 54.60 | 57.90 | 63.00 | 58.50 | 54.60 | 63.00 |
| 2.00 | 73.40 | 70.50 | 73.20 | 72.37 | 70.50 | 73.40 |
| 2.50 | 83.50 | 88.20 | 84.30 | 85.33 | 83.50 | 88.20 |
| 3.00 | 89.20 | 95.10 | 90.40 | 91.57 | 89.20 | 95.10 |
| 3.50 | 93.20 | 98.10 | 93.60 | 94.97 | 93.20 | 98.10 |
| 4.00 | 95.00 | 99.60 | 96.40 | 97.00 | 95.00 | 99.60 |
| 4.50 | 96.40 | 101.40 | 97.70 | 98.50 | 96.40 | 101.40 |
| 5.00 | 99.00 | 102.90 | 99.30 | 100.40 | 99.00 | 102.90 |
| 5.50 | 99.70 | 102.80 | 100.60 | 101.03 | 99.70 | 102.80 |
| 6.00 | 99.50 | 101.30 | 101.60 | 100.80 | 99.50 | 101.60 |

### In Vitro Examples

The following test was used to evaluate various ziprasidone formulations. First, for solid formulations (e.g., powders, beads), an amount of the formulation was accurately weighed into a glass dissolution flask such that the concentration of ziprasidone would have been 200 μgA/mL if all of the ziprasidone had dissolved, unless otherwise specified. The flask containing the formulation was then placed into a 37° C. water bath and 50 mL of 0.01 N HCl simulated gastric buffer at pH 2.0 was added to the flask. The flask was equipped with a stir paddle which was stirred at 100 rpm. The flask was also equipped with a UV probe which had been calibrated to ziprasidone standards of known concentration. A timer was set to zero when the simulated gastric buffer solution was added to the flask. The UV absorbance at 320 nm was then measured over time using the UV probes. From these data, the concentration of dissolved ziprasidone was calculated. The MDC and the AUC₉₀ were then determined from these data using standard techniques.

### Control 1

As a control, the dissolution performance of ziprasidone hydrochloride monohydrate of the form in GEODON/ZELDOX™ commercial capsules (i.e. crystalline ziprasidone hydrochloride monohydrate having a Volume Mean Diameter (VMD) particle size of about from about 5 microns to about 30 microns) was determined using the above test procedure. The MDC and AUC₉₀ are listed in Table 9.

TABLE 9

| | | MDC | | AUC |
|---|---|---|---|---|
| | | Enhancement | | Enhancement |
| | MDC | Relative to | AUC₉₀ | Relative to |
| Example | (μgA/mL) | Control 1 | (min-mg/mL) | Control 1 |
| Control 1 | 25 | — | 1.6 | — |
| 6 | 140 | 5.5 | 9.0 | 5.5 |
| 7 | 85 | 3.3 | 7.0 | 4.3 |
| 8 | 54 | 2.1 | 3.8 | 2.3 |
| 9 | 91 | 3.6 | 6.1 | 3.7 |
| 10 | 76 | 3.0 | 6.1 | 3.7 |
| 11 | 33 | 1.3 | 2.4 | 1.5 |
| 12 | 59 | 2.3 | 4.9 | 3.0 |
| 13 | 199 | 7.8 | 15.7 | 9.5 |
| 14 | 166 | 6.5 | 11.3 | 6.8 |
| 15 | 39 | 1.5 | 2.4 | 1.4 |
| 16 | 53 | 2.1 | 3.4 | 2.1 |
| 17 | 122 | 4.8 | 10.3 | 6.3 |

Various high-energy salt forms of ziprasidone were prepared using standard analytical techniques well known in the art and evaluated in the in vitro dissolution test described above. Example 6 consisted of ziprasidone mesylate, Example 7 consisted of ziprasidone free base, Example 8 consisted of ziprasidone tosylate, Example 9 consisted of ziprasidone tartrate, Example 10 consisted of ziprasidone aspartate, Example 11 consisted of ziprasidone citrate, and Example 12 consisted of ziprasidone succinate.

The results of these tests are presented in Table 9. These data show that the high energy salt forms of ziprasidone provided MDC values that were 1.3- to 5.5-fold that provided by Control 1, and AUC₉₀ values that were 1.5- to 5.5-fold that provided by Control 1, indicating these salt forms are dissolution-rate improved or solubility-improved forms of ziprasidone.

### Example 13

The form of ziprasidone described in Example 1 (ziprasidone-SBECD lyophilized complex) was evaluated in the dissolution test described above. The results of this test, summarized in Table 9, show that the lyophilized powder provided an MDC that was 7.8-fold that provided by Control 1, and an AUC₉₀ value that was 9.5-fold that provided by Control 1. Thus, the lyophilized powder is a dissolution-rate improved and/or solubility-improved form of ziprasidone.

### Example 14

The form of ziprasidone described in Example 2 (ziprasidone nanoparticles) was evaluated in the in vitro dissolution test as described above. The results of this test, summarized in Table 9, show that the ziprasidone nanoparticles provided an MDC that was 6.5-fold that provided by Control 1, and an AUC₉₀ value that was 6.8-fold that provided by Control 1. Thus, the ziprasidone nanoparticles are a dissolution-rate and/or solubility-improved improved form of ziprasidone.

### Example 15

A form of ziprasidone consisting of semi-ordered ziprasidone hydrochloride in an HPMCAS-H matrix, also known as a crystallized spray dried dispersion (CSDD) was prepared according to the following procedure. First, a spray solution was prepared consisting of 0.2 wt % ziprasidone hydrochloride monohydrate and 1 wt % HPMCAS-HG in methanol using the following procedure. Methanol was added to a stainless steel tank equipped with a top-mounted mixer. Next, the ziprasidone was added to the tank with agitation. The head space in the tank was purged with nitrogen to remove oxygen to prevent oxidative degradation of the ziprasidone. The tank was then heated to 50° C. to dissolve the ziprasidone. The HPMCAS was then added to the tank and mixed for 1 hour to form the spray solution, which was cooled to room temperature.

The spray solution was filtered through a 250 μm filter, and then pumped using a high-pressure pump to a spray drier (a Niro type XP Portable Spray-Dryer with a Liquid-Feed Process Vessel) (“PSD-1”), equipped with a pressure nozzle (SK 79-16 Pencil Point pressure nozzle). The PSD-1 was equipped with 9-inch chamber extension. The spray drier was also equipped with a DPH gas disperser for introduction of the drying gas to the spray drying chamber. The spray solution was pumped to the spray drier at about 120 g/min at a pressure of about 200 psi. Drying gas (e.g., nitrogen) was introduced to the spray drier through the DPH lid at a flow rate of about 1925 g/min and at an inlet temperature of about 150° C. The evaporated solvent and wet drying gas exited the spray drier at a temperature of about 53° C.

The resulting spray dried dispersion (SDD) was collected in a cyclone, and then dried in a convection tray drier with a powder depth of about 1 cm or less operating at 40° C. and 15% relative humidity (RH) for at least 4 hours. The CSDD was then formed in a tray drier by exposing the SDD to 50° C. and 90% RH for 24 hours. Analysis of the CSDD by PXRD, DSC, and TEM showed that the ziprasidone in the CSDD was semi-ordered, with crystal domain sizes on the order of 50 nm to 200 nm.

The resulting CSDD was evaluated in the in vitro dissolution test described above. The results of this test, summarized in Table 9, show that the 15% A CSDD provided an MDC that was 1.5-fold that provided by Control 1, and an AUC₉₀ value that was 1.4-fold that provided by Control 1. Thus, the 15% A CSDD formulation is a dissolution-rate improved and/or solubility-improved form of ziprasidone.

### Example 16

A form of ziprasidone consisting of a 25% A ziprasidone-hydrochloride:HPMCAS-H CSDD, was formed using the procedures outlined in Example 15 with the following exceptions: the spray solution consisted of 0.2 wt % ziprasidone hydrochloride monohydrate and 0.5 wt % HPMCAS-H in methanol. The resulting CSDD was evaluated in the in vitro dissolution test described above. The results of this test, summarized in Table 9, show that the 25% A CSDD provided an MDC that was 2.1-fold that provided by Control 1, and an AUC₉₀ value that was 2.1-fold that provided by Control 1. Thus, the 25% A CSDD formulation is a dissolution-rate improved and/or solubility-improved form of ziprasidone.

### Example 17

A form of ziprasidone consisting of 25% A ziprasidone-freebase:HPMC CSDD was formed using the following procedures. First, a spray solution was formed consisting of 0.85 wt % ziprasidone freebase, 2.55 wt % HPMC (E3 Premium LV), and 0.02 wt % butylated hydroxytoluene (BHT) in a 95/5 (w/w) tetrahydrofuran (THF)/water solvent as follows. First, the BHT was added to a stainless steel vessel equipped with a top-mounted mixer. The THF and water were then added to dissolve the BHT. The ziprasidone was then added to this mixture and the head-space purged with nitrogen to remove oxygen. The mixture was then mixed for at least 2 hours to dissolve the ziprasidone. The HPMC was then added to the mixture and mixed for at least 2 hours to form the spray solution.

The spray solution was filtered through a 250 μm filter, and then pumped using a high-pressure pump to a spray drier (a Niro type XP Portable Spray-Dryer with a Liquid-Feed Process Vessel) (“PSD-1”), equipped with a pressure nozzle (SK 78-16 Pencil Point pressure nozzle). The PSD-1 was equipped with 9-inch and 6 foot chamber extensions. The spray drier was also equipped with a DPH gas disperser for introduction of the drying gas to the spray drying chamber. The spray solution was pumped to the spray drier at about 131 g/min at a pressure of about 150 psi. Drying gas (e.g., nitrogen) was introduced to the spray drier through the DPH lid at a flow rate of about 1600 g/min and at an inlet temperature of about 106° C. The evaporated solvent and wet drying gas exited the spray drier at a temperature of about 42° C.

The resulting spray dried dispersion (SDD) was collected in a cyclone, and then dried in a convection tray drier with a powder depth of about 1 cm or less operating at 40° C. and 50% RH for at least 6 hours. The CSDD was then formed in a tray drier by exposing the SDD to 40° C. and 90% RH for 16 to 24 hours. Analysis of the CSDD by PXRD, DSC, and TEM showed that the ziprasidone in the CSDD was semi-ordered, with crystal domain sizes on the order of 50 nm to 200 nm.

The resulting CSDD was evaluated in the in vitro dissolution test described above. The results of this test, summarized in Table 9, show that the 25% A CSDD provided an MDC that was 4.8-fold that provided by Control 1, and an AUC₉₀ value that was 6.3-fold that provided by Control 1. Thus, the 25% A CSDD formulation is a dissolution-rate improved and/or solubility-improved form of ziprasidone.

### Examples 18-19

### Ziprasidone Hydrochloride Monohydrate CSDD Tablets (B1 and B2)

Immediate release tablets containing 40 mgA ziprasidone were prepared using the ziprasidone hydrochloride:HPMCAS-H CSDD described in Examples 15 and 16. The tablet composition for Example 18 was as follows:

| | | | Unit |
|---|---|---|---|
| | | | Formula |
| Ingredient | Grade | Function | (mg) |
| 25% A Ziprasidone | — | Active Ingredient | 160.07 |
| hydrochloride:HPMCAS-H | | | |
| CSDD (Example 16) | | | |
| Polyplasdone XL | NF | Disintegrant | 27.00 |
| Crospovidone | | | |
| Microcrystalline Cellulose | PhEurINF | Filler/Diluent | 87.64 |
| (Avicel PH102) | | | |
| Lactose Monohydrate (Fast Flo | PhEurINF | Filler/Diluent | 175.29 |
| 316) | | | |
| Magnesium Stearate | NF | Tableting Aid | (trace) |
| Core Tablet Weight | | | 450.00 |

The tablet composition for Example 19 was as follows:

| | | | Unit |
|---|---|---|---|
| | | | Formula |
| Ingredient | Grade | Function | (mg) |
| 15% A Ziprasidone | — | Active Ingredient | 276.22 |
| hydrochloride:HPMCAS-H | | | |
| CSDD (Example 15) | | | |
| Polyplasdone XL | NF | Disintegrant | 30.00 |
| Crospovidone | | | |
| Microcrystalline Cellulose | PhEurINF | Filler/Diluent | 64.60 |
| (Avicel PH102) | | | |
| Lactose Monohydrate (Fast Flo | PhEurINF | Filler/Diluent | 129.18 |
| 316) | | | |
| Magnesium Stearate | NF | Tableting Aid | (trace) |
| Core Tablet Weight | | | 500.00 |

### Examples 20-22

### Ziprasidone Hydrochloride Monohydrate CSDD Matrix Tablets—Short (B3), Medium (B4), Long Duration

Sustained release matrix tablets, each containing 40 mgA ziprasidone, were prepared using the 25% A ziprasidone hydrochloride:HPMCAS-H CSDD described in Example 16. The tablet composition for Example 20 (B3) was as follows:

| | | | | | |
|---|---|---|---|---|---|
| Item # | Component | Use | Grade | Wt % | mg/tablet |

Intragranular Components

| | | | | | |
|---|---|---|---|---|---|
| 1 | 25% A ziprasidone | Active | Pharm | 30.00 | 160.00 |
| | HCl: HPMCAS | | | | |
| | CSDD | | | | |
| 2 | Hypromellose, | Matrix | USP | 17.00 | 90.67 |
| | Methocel K100LV | Polymer | | | |
| | Premium CR | | | | |
| 3 | Magnesium Stearate | Lubricant | NF | 0.25 | 1.33 |

Extragranular Components

| | | | | | |
|---|---|---|---|---|---|
| 4 | Lactose, Fast Flo | Diluent | NF | 52.50 | 280.00 |
| | 316 Spray Dried | | | | |
| 5 | Magnesium Stearate | Lubricant | NF | 0.25 | 1.33 |

| | | |
|---|---|---|
| Total | 100.00 | 533.33 |

The tablet composition for Example 21 (B4) was as follows:

| | | | | | |
|---|---|---|---|---|---|
| | | | | | mg/ |
| Item # | Component | Use | Grade | Wt % | tablet |

Intragranular Components

| | | | | | |
|---|---|---|---|---|---|
| 1 | 25% A ziprasidone | Active | Pharm | 30.00 | 160.00 |
| | HCl: HPMCAS CSDD | | | | |
| 2 | Hypromellose, | Matrix | USP | 25.00 | 133.33 |
| | Methocel K100LV | Polymer | | | |
| | Premium CR | | | | |
| 3 | Magnesium Stearate | Lubricant | NF | 0.25 | 1.33 |

Extragranular Components

| | | | | | |
|---|---|---|---|---|---|
| 4 | Lactose, Fast Flo 316 | Diluent | NF | 44.50 | 237.34 |
| | Spray Dried | | | | |
| 5 | Magnesium Stearate | Lubricant | NF | 0.25 | 1.33 |

| | | |
|---|---|---|
| Total | 100.00 | 533.33 |

The tablet composition for Example 22 (long-duration tablet) was as follows:

| | | | | | |
|---|---|---|---|---|---|
| | | | | | mg/ |
| Item # | Component | Use | Grade | Wt % | tablet |

Intragranular Components

| | | | | | |
|---|---|---|---|---|---|
| 1 | 25% A ziprasidone | Active | Pharm | 30.00 | 160.00 |
| | HCl: HPMCAS CSDD | | | | |
| 2 | Hypromellose, | Matrix | USP | 30.00 | 160.00 |
| | Methocel K4M | Polymer | | | |
| | Premium CR | | | | |
| 3 | Magnesium Stearate | Lubricant | NF | 0.25 | 1.33 |

Extragranular Components

| | | | | | |
|---|---|---|---|---|---|
| 4 | Lactose, Fast Flo 316 | Diluent | NF | 39.50 | 210.67 |
| | Spray Dried | | | | |
| 5 | Magnesium Stearate | Lubricant | NF | 0.25 | 1.33 |

| | | |
|---|---|---|
| Total | 100.00 | 533.33 |

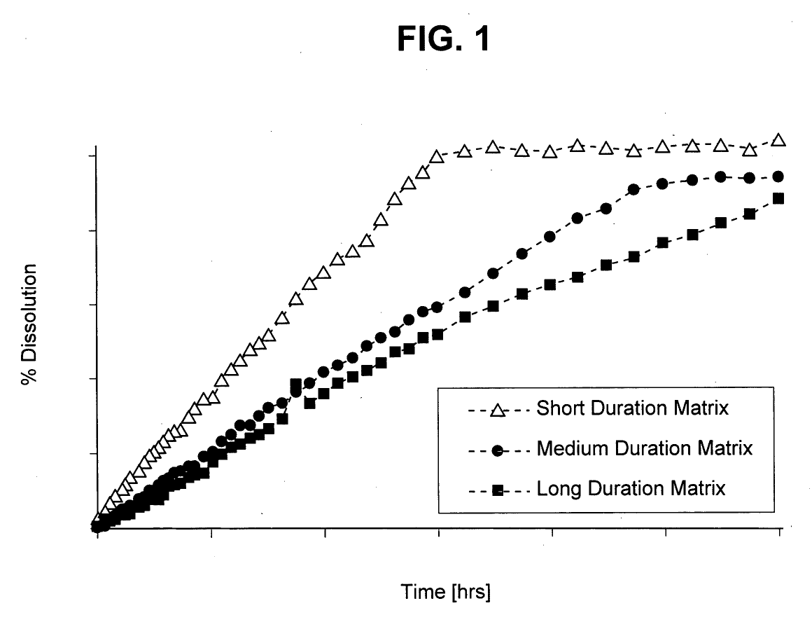

In vitro dissolution test results for Examples 20, 21 and 22 are shown in FIG. 1.

### Examples 23-25

### 15% A Ziprasidone Hydrochloride Monohydrate CSDD Matrix Tablets—Short, Medium, Long Duration

Sustained release matrix tablets, each containing 40 mgA ziprasidone, were prepared using the 15% A ziprasidone hydrochloride:HPMCAS-H CSDD described in Example 15. The tablet composition for Example 23 (short duration tablet) was as follows:

| | | | | | |
|---|---|---|---|---|---|
| | | | | | mg/ |
| Item # | Component | Use | Grade | Wt % | tablet |

Intragranular Components

| | | | | | |
|---|---|---|---|---|---|
| 1 | 15% A ziprasidone | Active | Pharm | 30.00 | 266.67 |
| | HCl: HPMCAS CSDD | | | | |
| 2 | Hypromellose, | Matrix | USP | 12.00 | 106.67 |
| | Methocel K4M | Polymer | | | |
| | Premium CR | | | | |
| 3 | Magnesium Stearate | Lubricant | NF | 0.25 | 2.22 |

Extragranular Components

| | | | | | |
|---|---|---|---|---|---|
| 4 | Lactose, Fast Flo 316 | Diluent | NF | 57.50 | 511.11 |
| | Spray Dried | | | | |
| 5 | Magnesium Stearate | Lubricant | NF | 0.25 | 2.22 |

| | | |
|---|---|---|
| Total | 100.00 | 888.89 |

The tablet composition for Example 24 (medium duration tablet) was as follows:

| | | | | | |
|---|---|---|---|---|---|
| | | | | | mg/ |
| Item # | Component | Use | Grade | Wt % | tablet |

Intragranular Components

| | | | | | |
|---|---|---|---|---|---|
| 1 | 15% A ziprasidone | Active | Pharm | 30.00 | 266.67 |
| | HCl: HPMCAS | | | | |
| | CSDD | | | | |
| 2 | Hypromellose, | Matrix | USP | 20.00 | 177.78 |
| | Methocel K100LV | Polymer | | | |
| | Premium CR | | | | |
| 3 | Magnesium Stearate | Lubricant | NF | 0.25 | 2.22 |

Extragranular Components

| | | | | | |
|---|---|---|---|---|---|
| 4 | Lactose, Fast Flo 316 | Diluent | NF | 49.50 | 440.00 |
| | Spray Dried | | | | |
| 5 | Magnesium Stearate | Lubricant | NF | 0.25 | 2.22 |

| | | |
|---|---|---|
| Total | 100.00 | 888.89 |

The tablet composition for Example 25 (long-duration tablet) was as follows:

| | | | | | |
|---|---|---|---|---|---|
| | | | | | mg/ |
| Item # | Component | Use | Grade | Wt % | tablet |

Intragranular Components

| | | | | | |
|---|---|---|---|---|---|
| 1 | 15% A ziprasidone | Active | Pharm | 30.00 | 266.67 |
| | HCl: HPMCAS | | | | |
| | CSDD | | | | |
| 2 | Hypromellose, | Matrix | USP | 20.00 | 177.78 |
| | Methocel K4M | Polymer | | | |
| | Premium CR | | | | |
| 3 | Magnesium Stearate | Lubricant | NF | 0.25 | 2.22 |

Extragranular Components

| | | | | | |
|---|---|---|---|---|---|
| 4 | Lactose, Fast Flo 316 | Diluent | NF | 49.50 | 440.00 |
| | Spray Dried | | | | |
| 5 | Magnesium Stearate | Lubricant | NF | 0.25 | 2.22 |

| | | |
|---|---|---|
| Total | 100.00 | 888.89 |

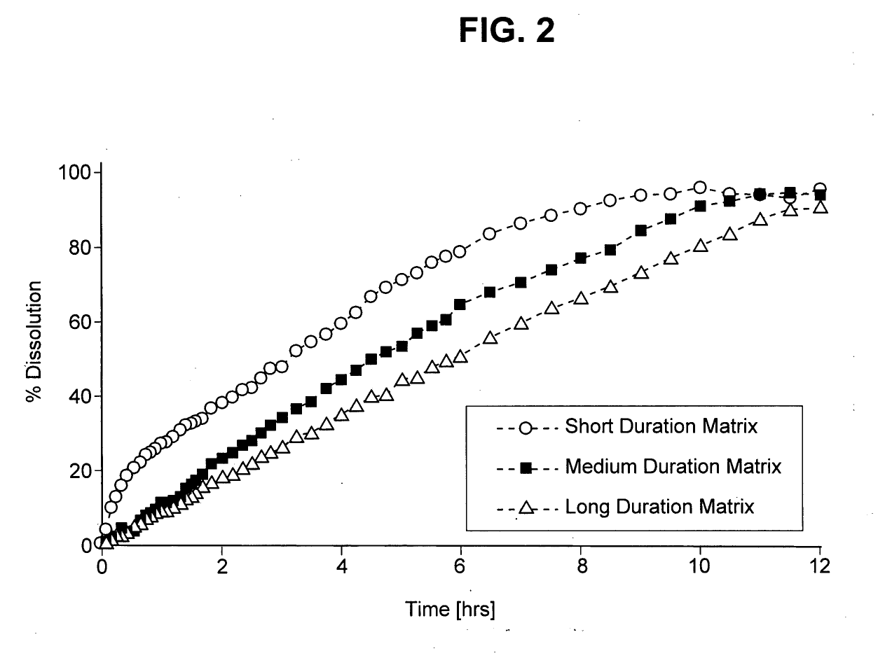

In vitro dissolution test results for Examples 23, 24 and 25 in 0.05M NaH2PO4 media with the addition of 2% (w/v) sodium dodecyl sulfate (SDS) and adjusted to a pH of 7.5, paddles at 75 rpm are shown in FIG. 2.

### Example 26

### Ziprasidone Free Base CSDD Tablet (D1)

An immediate release dosage form containing 40 mgA ziprasidone was prepared using the 25% A ziprasidone free base HPMC CSDD formulation described in Example 17. The formulation for the tablet is as follows:

| Component | Mass in Tablet (mg) |
|---|---|
| 25% A Ziprasidone free base HPMC CSDD | 160.00 |
| Polyplasdone XL Crospovidone | 27.00 |
| Microcrystalline cellulose (AVICEL | 87.67 |
| PH102) | |
| Lactose monohydrate (FAST FLO 316) | 175.33 |
| Magnesium stearate | (trace) |
| Total | 450.00 |

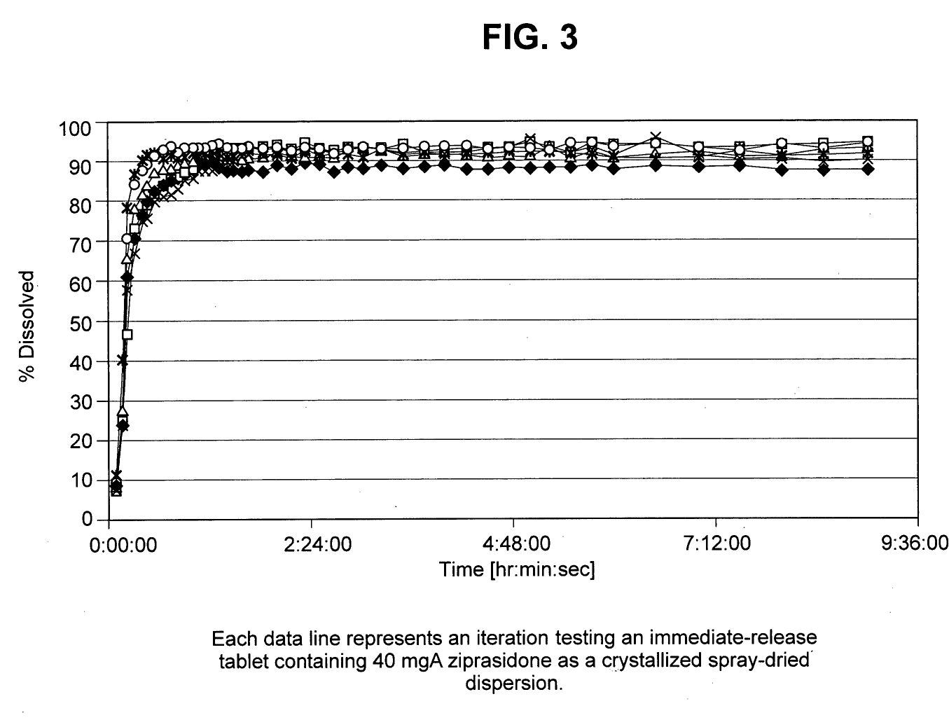

In vitro dissolution test results for Formulation D1 is as shown in FIG. 3.

### Example 27

### Coated Beads

Ziprasidone coated beads were prepared using the following procedures. First, a solution was formed consisting of 26.67 wt % of the 25% A ziprasidone hydrochloride:HPMCAS-H CSDD described in Example 16 and 3.33 wt % HPMC E3 Premium in water, forming a suspension of the CSDD particles. This solution was then wet-milled using a Willy A Bachofen (WAB) DynoMill model KDL agitator-bead mill in a single-pass configuration. The milling chamber had a volume of 0.3 L, and was equipped with a gap separator of 0.15 mm. The grinding beads were 0.7 to 1.0 mm lead-free glass, and had a bulk volume of 250 mL. The suspension was cooled to 5° C. during the milling process. The mill speed was 4200 rpm, and the milling time was 29 min/kg suspension. The resulting milled suspension was diluted with water containing dissolved HPMC to form a spray suspension consisting of 12 wt % CSDD, 3 wt % HPMC, and water.

The spray suspension, was then coated onto 20/25 mesh sugar spheres using a Niro MP-2 fluid-bed with a reduced-bowl (e.g. MP-1) Precision Coater insert to a 55 wt % coating weight level. The coating conditions were as follows. The fluidized bed was equipped with an 80-mm column, with a 250-mm height; a 30-mm swirl insert, and a 20-mm partition height gap. The spray nozzle was a Schlick 970 with a 1.2-mm insert and a 3.0-mm spacer. The fluidizing air was set at an inlet temperature of 65° C., a dew point temperature of 11° C., and a flow rate of 90 m³/hr. The bed temperature was kept at 32° C. The spray suspension feed rate was 16 g/min, and the atomization pressure was 1.5 barg.

The resulting ziprasidone-coated beads consisted of 44 wt % CSDD, 11 wt % HPMC, and 45 wt % sugar spheres, and contained about 11 wt % A ziprasidone.

### Example 28

### Eudragit® Enteric Coated Beads (Form B6)

The ziprasidone-coated beads of Example 27 were coated with an enteric polymer in a fluidized bed process. The enteric, coating solution consisted of 15.84 wt % Eudragit®-L30D-55, 1.76 wt % triethylcitrate, and 82.40 wt % water. This enteric coating solution was sprayed onto the ziprasidone-coated beads to achieve a coating weight of 10.1 wt % using the same conditions used in Example 24 for spray coating the CSDD onto the beads. The resulting enteric coated beads consisted of about 9.89 wt % A ziprasidone.

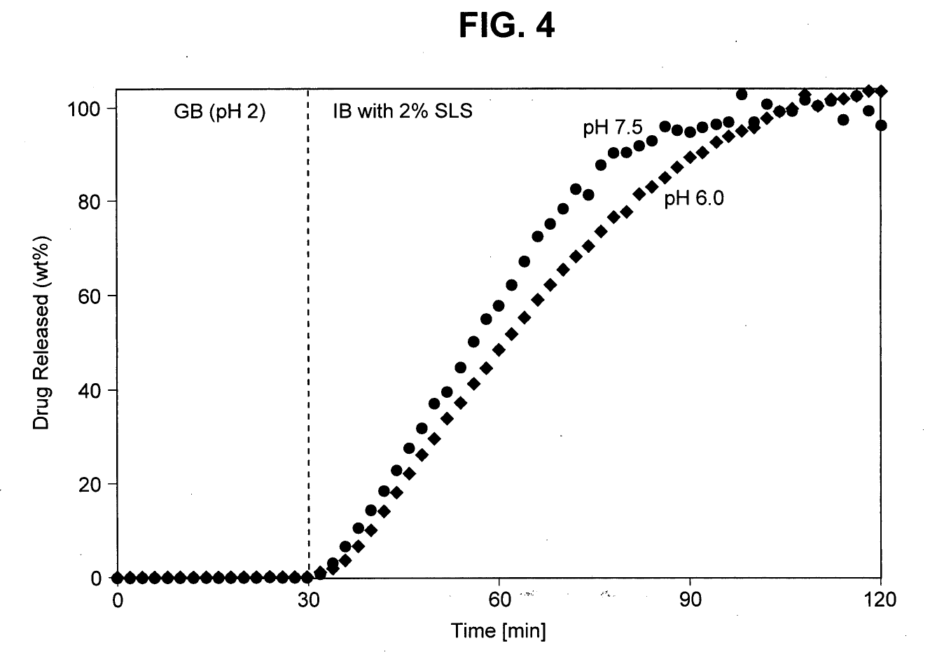

In vitro dissolution test results for Formulation B6 are shown in FIG. 4. pH 6.0 is represented by diamonds; pH 7.5 is represented by circles.

### Example 29

### HPMCAS-H Enteric Coated Beads (Form B5)

The ziprasidone-coated beads of Example 27 were coated with an enteric polymer in a fluidized bed process. The enteric coating solution consisted of 8 wt % HPMCAS-H and 92 wt % acetone. This enteric coating solution was sprayed onto the ziprasidone-coated beads to achieve a coating weight of 20 wt % using the same conditions used in Example 24 for spray coating the CSDD onto the beads. The resulting enteric coated beads consisted of about 8.8 wt % A ziprasidone.

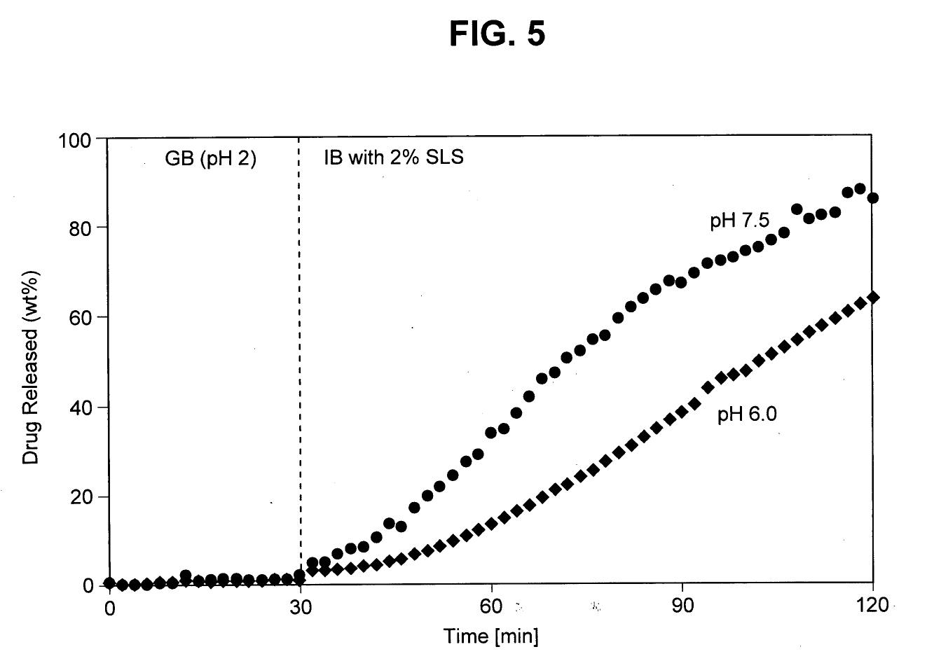

In vitro dissolution test results for Formulation B5 are shown in FIG. 5. pH 6.0 is represented by diamonds; pH 7.5 is represented by circles.

1. A method for treating a CNS disorder in a human, which method comprises administering to the human in a fasted state, a solid oral dosage form comprising an amount of ziprasidone effective to treat said CNS disorder, wherein the area under the serum concentration versus time curve (AUC_{0-inf}) of the ziprasidone in the human subsequent to said administering is from 70% to 140% of the mean area under the ziprasidone serum concentration versus time curve (AUC_{0-inf}) resulting from administration of a control ziprasidone immediate release oral capsule containing the same amount of ziprasidone to a cohort of humans in a fed state.

2. A method for treating a CNS disorder in a human, which method comprises administering to the human in a fasted state a solid oral dosage form comprising an amount of ziprasidone effective to treat said CNS disorder, wherein the area under the serum concentration versus time curve (AUC_{0-inf}) of the ziprasidone in the human subsequent to said administering is from 70% to 140% of the mean area under the ziprasidone serum concentration versus time curve (AUC_{0-inf}) resulting from administering an identical solid oral dosage form, containing the same amount of ziprasidone, to a cohort of humans in a fed state.

3. (canceled)

4. A method according to claim 1, wherein the maximum ziprasidone serum concentration (Cₘₐₓ) subsequent to administration in the fasted state is less than about 140% of the maximum ziprasidone serum concentration (Cₘₐₓ) resulting from administration of a control ziprasidone immediate release oral capsule containing the same amount of ziprasidone to a human in a fed state.

5. A method for treating a CNS disorder in a human, which method comprises administering to the human in a fasted state a solid oral dosage form comprising an effective amount of ziprasidone providing to the human in the fasted state a steady state minimum blood ziprasidone concentration (Cₘᵢₙ) of at least 20 ng/ml and a steady state maximum blood ziprasidone concentration (Cₘₐₓ) of less than 330 ng/ml.

6. A method according to any one of claim 1, wherein the ziprasidone in the dosage form comprises ziprasidone in a dissolution rate-improved form or a solubility-improved form and/or ziprasidone in combination with a precipitation inhibitor.

7. A method according to claim 6 wherein the ziprasidone in the dosage form comprises ziprasidone tosylate, ziprasidone tartrate, or ziprasidone in combination with a cyclodextrin.

8. A method according to claim 6 wherein the ziprasidone in the dosage form comprises ziprasidone nanoparticles.

9. A method according to claim 6 wherein the ziprasidone in the dosage form comprises a solid mixture of ziprasidone and a polymer, at least a portion of which ziprasidone in the solid mixture is in a semi-ordered state.

10. A method according to claim 6, wherein the solid oral dosage form comprises a sustained release means, a delayed release means, an immediate release portion, or any combination thereof.

11. A method according to any one of claim 1, wherein the solid oral dosage form comprises a sustained release means, and optionally a delayed release means and/or an immediate release portion, and wherein the ziprasidone in the solid oral dosage form comprises a solid mixture of ziprasidone with a polymer, at least a portion of which ziprasidone is in a semi-ordered state.

12. A method according to any one of claim 1, wherein the solid oral dosage form is an immediate release tablet or a sustained release tablet, said tablet comprising a solid mixture comprising semi-ordered ziprasidone hydrochloride and hydroxypropyl methylcellulose acetate succinate (HPMCAS).

13. A method according to any one of claim 1 wherein the solid oral dosage form comprises an immediate-release portion and a sustained release portion, wherein the sustained release portion comprises a solid mixture comprising semi-ordered ziprasidone hydrochloride and HPMCAS.

14.-16. (canceled)

17. A kit comprising
a) a solid oral dosage form comprising an effective amount of ziprasidone and a pharmaceutically acceptable carrier; and
b) instructions for oral administration of the dosage form of (a), which
i) do not specify administration with food, or
ii) indicate that the dosage form of (a) may be administered with or without food;
wherein said solid oral dosage form when administered to a human in a fasted state provides to the human a serum ziprasidone AUC_{0-inf} which is from 70% to 140% of a mean ziprasidone serum AUC_{0-inf} resulting from administration of an identical ziprasidone solid oral dosage form containing the same amount of ziprasidone to a cohort of humans in a fed state.

18. A kit according to claim 16, wherein the ziprasidone comprises ziprasidone in a dissolution rate-improved form or a solubility-improved form and/or ziprasidone in combination with a precipitation inhibitor.

19. A solid oral dosage form comprising a pharmaceutically acceptable carrier and an amount of ziprasidone effective to treat a CNS disorder, which dosage form comprises ziprasidone in a dissolution rate-improved form or a solubility-improved form, and which dosage form provides to a human in a fasted state an area under the serum concentration versus time curve (AUC_{0-inf}) of ziprasidone that is from 70% to 140% of the mean area under a ziprasidone serum concentration versus time curve (AUC_{0-inf}) resulting from administering an identical solid oral dosage form, containing the same amount of ziprasidone, to a cohort of humans in a fed state.

20. A solid oral dosage form according to claim 19, wherein the amount of ziprasidone in the dosage form is 20, 40, 60 or 80 mgA.

21. A solid oral dosage form according to claim 19, wherein the ziprasidone in the dosage form comprises a solid mixture of ziprasidone and a polymer, at least a portion of which ziprasidone in the solid mixture is in a semi-ordered state.

22. A solid oral dosage form according to claim 21, wherein the polymer is sleeted from the group consisting of hydroxypropyl methyl cellulose acetate succinate, hydroxypropyl methyl cellulose phthalate, hydroxypropyl methyl cellulose, cellulose acetate phthalate (CAP), cellulose acetate trimellitate, carboxymethyl ethylcellulose, poloxamers, polyvinyl pyrrolidone (PVP), and mixtures thereof.

23. A solid oral dosage form according to claim 21, wherein the polymer is hydroxypropyl methylcellulose acetate succinate.

24. A solid oral dosage form according to claim 19, wherein the solid oral dosage form comprises a sustained release means, and optionally a delayed release means and/or an immediate release portion.

25. A solid oral dosage form according to claim 19, wherein the solid oral dosage form is an immediate release tablet or a sustained release tablet, said tablet comprising a solid mixture comprising semi-ordered ziprasidone hydrochloride and hydroxypropyl methylcellulose acetate succinate.

26. A solid oral dosage form according to claim 19, wherein the solid oral dosage form comprises an immediate-release portion and a sustained release portion, wherein the dosage form comprises a solid mixture comprising semi-ordered ziprasidone hydrochloride and hydroxypropyl methylcellulose acetate succinate.

27. A solid oral dosage form according to claim 19, wherein the solid oral dosage form comprises an immediate-release portion and a delayed release portion, wherein the dosage form comprises a solid mixture comprising semi-ordered ziprasidone hydrochloride and hydroxypropyl methylcellulose acetate succinate.

28. A solid oral dosage form according to claim 19, wherein the solid oral dosage form is an osmotic tablet or matrix tablet.
